(19) United States
(12) Patent Application Publication
(10) Pub. No.: US 2026/0054038 A1
(43) Pub. Date: Feb. 26, 2026

(54) WIRE AND CATHETER PLACEMENT DEVICE

(71) Applicant: Main Circle Medical Inc., Charlotte, NC (US)

(72) Inventor: Kyle Murphy, Lexington, KY (US)

(21) Appl. No.: 19/374,500

(22) Filed: Oct. 30, 2025

Related U.S. Application Data
(63) Continuation of application No. PCT/US2025/043725, filed on Aug. 27, 2025.
(63) Continuation-in-part of application No. 17/550,483, filed on Dec. 14, 2021, now Pat. No. 12,465,729.
(60) Provisional application No. 63/125,913, filed on Dec. 15, 2020.
(60) Provisional application No. 63/215,493, filed on Jun. 27, 2021.
(60) Provisional application No. 63/687,698, filed on Aug. 27, 2024.

(51) Int. Cl. A61M 25/09; A61M 25/06

(52) U.S. Cl. CPC A61M 25/09041; A61M 25/065; A61M 2205/582; A61M 2205/8281

(57) ABSTRACT

A device for accessing a tissue workspace and delivering a wire into the tissue workspace includes a housing providing a syringe body and a plunger movable in the syringe body. A slide couples with the syringe plunger for moving together with the syringe plunger between a forward position and a retracted position when the device is activated. A needle is configured for coupling with the housing and has a bore for guiding a proximal end of the wire into the workspace. The slide couples with a distal end of the wire for moving the distal end and advancing the proximal end of the wire forwardly in the needle when the slide moves to a retracted position. A bias force element, such as a coil spring, is coupled between the housing and the slide. The bias force element is configured for biasing the slide toward the retracted position when the device is activated.

### CROSS-REFERENCE TO RELATED APPLICATIONS

This application is a Continuation-in-Part application of U.S. patent application Ser. No. 17/550,483 entitled Wire and Catheter Placement Device, filed Dec. 14, 2020, which U.S. application claims the benefit of priority to U.S. Provisional Patent Application Ser. No. 63/125,913, filed Dec. 15, 2020, and to U.S. Provisional Patent Application Ser. No. 63/215,493, filed Jun. 27, 2021. This application is also a Continuation application of PCT/US2025/043725 entitled Access Device for Accessing Vascular Cavities and Other Tissue Cavities, filed Aug. 27, 2025, which PCT application claims the benefit of priority to U.S. Provisional Patent Application Ser. No. 63/687,698, filed Aug. 27, 2024, the disclosures of which applications are all incorporated by reference herein, in their entireties.

### TECHNICAL FIELD OF THE INVENTION

The present invention is directed to a device for positioning a guide wire into a workspace inside tissue cavities, such as vascular tissue, for providing access into the tissue for the positioning of other elements for the performance of medical procedures.

### BACKGROUND OF THE INVENTION

Commonly in medicine and surgery, it is necessary to position elements, such as catheters and other elements, into workspaces or cavities within tissue. One such area is in the cavities of vascular tissue. The Seldinger technique is often used for such positioning of a guide wire. Typically in such a technique, a needle is inserted into a vein, artery, or other body tissue cavity or workspace. Frequently, a syringe is attached to the needle, and a user draws the syringe back to generate negative pressure or a vacuum as the needle is advanced into skin and tissue cavity. While the needle goes through the skin and subcutaneous tissue, the lumen or bore of the needle is blocked and a user is only able to pull the plunger back a short distance due to the vacuum and negative pressure created within the syringe. Once the needle tip or bore enters the target cavity space, such as the inside of a blood vessel, the syringe draws in blood, air, or another fluid under the force of the negative pressure. A user inserting the needle is able to visualize and feel this to know the needle is properly placed in the cavity.

Once the needle tip is in the target cavity space, the syringe is removed from the needle. From there, a flexible wire is passed through the needle into the target space. The needle then can be slid back and removed over the wire. A dilator, which is a firm piece of plastic, is passed over the wire into the tissue and workspace and then removed. A catheter, which is generally the same size as the dilator but more flexible, can then be placed into the target space over the wire, being guided into the cavity by the wire.

One of the current drawbacks to the existing technique is that several issues can arise at the various stages, from the removal of the syringe from the needle to the feeding of the wire into the needle and into the workspace. For a user, moving and manipulating the wire can be awkward and difficult. The wire is thin and frequently flops around. As such, the wire can become nonsterile if part of it hits something outside of the sterile field. It can also be difficult to get the wire to go into the back of the needle. The needle can also move while the syringe is removed therefrom and while a user reaches for the wire and threads it into the needle. In such a scenario, the needle can either pull out of the target space so that the wire cannot be advanced. The needle might also be advanced further than desired. This may create complications. For example, in the case of an internal jugular central venous catheter placement, the carotid artery is often posterior. If the needle goes in further than desired in the vein, the needle tip can then be in an artery without the user knowing it. The wire is passed into the artery. The artery might then be dilated. This can lead to severe complications, given that a larger hole has just been made in a high-pressure arterial system.

Accordingly, there is a need for a device and method that can improve safety and time efficiency in the Seldinger technique and similar techniques for positioning a wire in a body tissue workspace or cavity. There is also a need to improve ergonomics and allow a user to more easily draw negative pressure and receive feedback in the process during wire placement. There is still further a need to create the ability to place the wire with one hand while managing another task with the other hand of the user.

### SUMMARY OF THE INVENTION

A device for accessing a tissue workspace and delivering a wire into the tissue workspace includes a housing providing a syringe body and a plunger movable in the syringe body. A slide is configured for coupling with the syringe plunger for moving together with the syringe plunger between a forward position and a retracted position when the device is activated. The slide is coupled to the wire. A needle is configured for coupling with the housing and has at least one bore for guiding a proximal end of the wire into the workspace. The slide is configured for coupling with a distal end of the wire for moving the distal end and advancing the proximal end of the wire forwardly in the needle when the slide moves together with the syringe plunger from the forward position to the retracted position. A bias force element, such as a coil spring, is coupled between the housing and the slide. The bias force element is configured for biasing the slide toward the retracted position when the device is activated.

### BRIEF DESCRIPTION OF THE DRAWINGS

The accompanying drawings, which are incorporated in and constitute a part of this specification, illustrate embodiments of the invention and, together with a general description of the invention given below, serve to explain the principles of the invention.

FIG. 1 is a perspective view of wire and catheter placement device in accordance with an embodiment of the invention.

FIG. 1A is an exploded, perspective view of wire and catheter placement device in accordance with an embodiment of the invention.

FIG. 2A is a side view of the wire and catheter placement device of FIG. 1, in a forward position, in accordance with an embodiment of the invention.

FIG. 2B is a side view of the wire and catheter placement device of FIG. 1, in a retracted position, in accordance with an embodiment of the invention.

FIG. 3A is a top view, in partial cross-section, of the wire and catheter placement device of FIG. 1, in a forward position, in accordance with an embodiment of the invention.

FIG. 3B is a top view, in partial cross-section, of the wire and catheter placement device of FIG. 1, in a retracted position, in accordance with an embodiment of the invention.

FIG. 4A is a side view of the wire and catheter placement device of FIG. 1, in a forward position, inserting a wire into tissue.

FIG. 4B is a side view of the wire and catheter placement device of FIG. 1, in a retracted position, inserting a wire into a workspace in tissue.

FIG. 4C is a side view of the wire and catheter placement device of FIG. 1, showing a catheter arrangement for insertion into a workspace in tissue via a wire.

FIG. 4D is a side view showing a catheter arrangement for insertion into a workspace in tissue via a wire inserted in accordance with an embodiment of the invention.

FIG. 4E is a side view showing a catheter arrangement inserted into a workspace in tissue via a wire inserted in accordance with an embodiment of the invention.

FIG. 4F is a side view showing a catheter inserted into a workspace in tissue via a wire inserted in accordance with an embodiment of the invention.

FIG. 5 is a perspective view of wire and catheter placement device in accordance with another embodiment of the invention.

FIG. 6A is a side view of the wire and catheter placement device of FIG. 5, in a forward position, with a wire and catheter arrangement for placement in the device.

FIG. 6B is a side view of the wire and catheter placement device of FIG. 5, in a forward position, with a wire and catheter arrangement in the device.

FIG. 6C is a side view of the wire and catheter placement device of FIG. 5, moving to a retracted position, to advance a wire for placement of the catheter arrangement.

FIG. 6D is a side view of the wire and catheter placement device of FIG. 5, in a retracted position, with an advanced wire for placement of the catheter arrangement.

FIG. 6E is a top view, in partial cross-section, of the wire and catheter placement device of FIG. 5, in a retracted position, with an advanced wire for placement of the catheter arrangement.

FIG. 6F is a top view, in partial cross-section, of the wire and catheter arrangement of FIG. 6E, with most of the wire and catheter placement device of FIG. 5 removed.

FIG. 6G is a top view, in partial cross-section, of the wire and catheter arrangement of FIG. 6F to be inserted into a workspace in tissue via a wire inserted in accordance with an embodiment of the invention.

FIG. 6H is a top view, in partial cross-section, of the wire and catheter arrangement of FIG. 6G to be inserted into a workspace in tissue, with a removal of the needle.

FIG. 6I is a top view, in partial cross-section, of the wire and catheter arrangement of FIG. 6G inserted into a workspace in tissue.

FIG. 6J is a top view, in partial cross-section, of the catheter arrangement of FIG. 6I inserted into a workspace in tissue, with a dilator removed

FIG. 7 is a perspective view of wire and catheter placement device in accordance with another embodiment of the invention.

FIG. 8A is a top view, in partial cross-section, of the wire and catheter placement device of FIG. 7, in a forward position, in accordance with an embodiment of the invention.

FIG. 8B is a top view, in partial cross-section, of the wire and catheter placement device of FIG. 7, in a retracted position, in accordance with an embodiment of the invention.

FIG. 9 is a perspective view of wire and catheter placement device in accordance with another embodiment of the invention.

FIG. 10 is a side view of the wire and catheter placement device of, in a forward position.

FIG. 11 is a perspective view of an alternative needle for the wire and catheter placement device of an embodiment of the invention.

FIG. 12 is another perspective view of the alternative needle of FIG. 11.

FIG. 13 is a top view of the alternative needle of FIG. 11.

FIG. 14 is a top view of an alternative device of the invention.

FIG. 15 is a perspective view of a vascular access device in accordance with another embodiment of the invention.

FIG. 16 is another perspective view of a vascular access device in accordance with the embodiment of FIG. 15.

FIG. 17 is an exploded, perspective view of a vascular access device in accordance with the embodiment of FIG. 15.

FIG. 18A is a side view, in partial cross-section, of the vascular access device of FIG. 15, in a released position, in accordance with the embodiment of the FIG. 18B is a side view, in partial cross-section, of the vascular access device of FIG. 15, in a primed position, in accordance with the embodiment of the invention.

FIG. 18C is a side view, in partial cross-section, of the vascular access device of FIG. 15, in an activated position, in accordance with the embodiment of the invention.

FIG. 18D is a side view, in partial cross-section, of the vascular access device of FIG. 15, again in a released position after activation, in accordance with the embodiment of the invention.

FIG. 19A is a sectional side view, in partial cross-section, of the activation mechanism of the vascular access device of FIG. 15, in a released position, in accordance with the embodiment of the invention.

FIG. 19B is a sectional side view, in partial cross-section, of the activation mechanism of the vascular access device of FIG. 15, in a position to be primed, in accordance with the embodiment of the invention.

FIG. 19C is a sectional side view, in partial cross-section, of the activation mechanism of the vascular access device of FIG. 15, in a primed position, in accordance with the embodiment of the invention.

FIG. 19D is a sectional side view, in partial cross-section, of the activation mechanism of the vascular access device of FIG. 15, in a partially activated position, in accordance with the embodiment of the invention.

FIG. 19E is a sectional side view, in partial cross-section, of the activation mechanism of the vascular access device of FIG. 15, in an activated position, in accordance with the embodiment of the invention.

FIG. 19F is a sectional side view, in partial cross-section, of the activation mechanism, in a partially activated position, in accordance with an alternative embodiment of the invention.

FIG. 20A is a side view of the vascular access device of FIG. 15, in a primed position and deployed in vascular tissue, in accordance with an embodiment of the invention.

FIG. 20B is a side view of the vascular access device of FIG. 15, in an activated and released position and deployed in vascular tissue, in accordance with an embodiment of the invention.

FIG. 21 is a side view, in cross-section, of the vascular access device, in a released position, in accordance with an alternative embodiment of the invention.

FIG. 22 is a side view of an embodiment of a wire used in accordance with an embodiment of the invention.

FIG. 22A is a side view, in cross-section, of another wire used in accordance with an embodiment of the invention.

FIG. 22B is a side view, in cross-section, of another wire used in accordance with an embodiment of the invention.

FIG. 22C is a side view, in cross-section, of another wire used in accordance with an embodiment of the invention.

FIG. 22D is a side view, in cross-section, of another wire used in accordance with an embodiment of the invention.

FIG. 23 is a side view, in cross-section, of the vascular access device, in a primed position, in accordance with an alternative embodiment of the invention.

FIG. 24 is a side view, in cross-section, of the vascular access device, in a primed position, in accordance with an alternative embodiment of the invention.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION OF EMBODIMENTS OF THE INVENTION

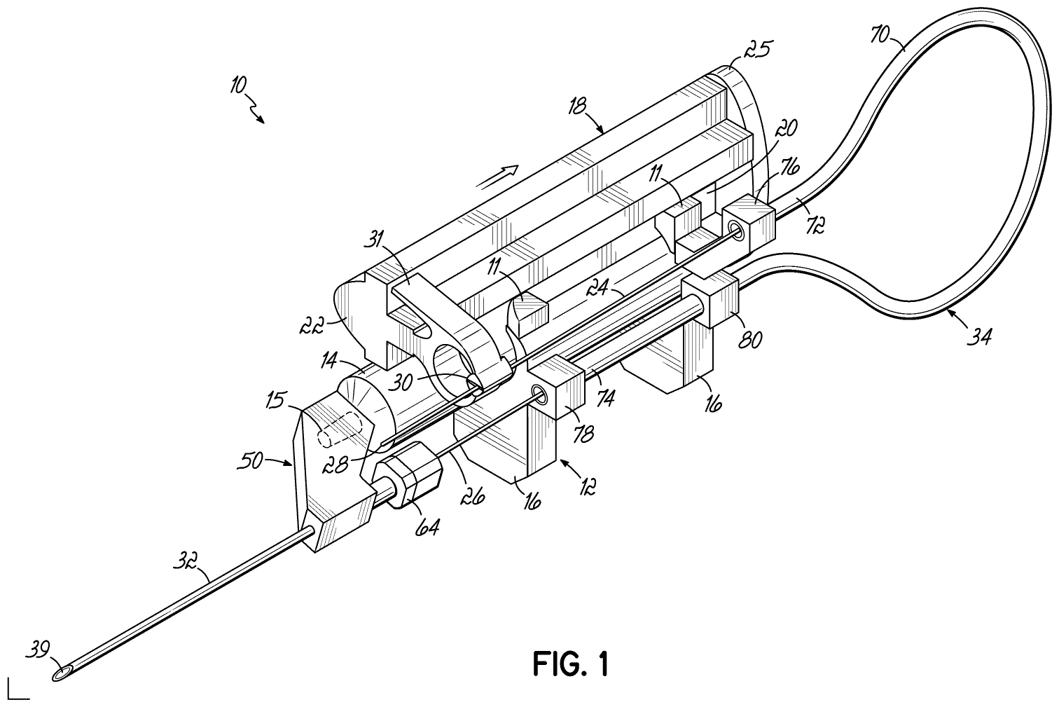

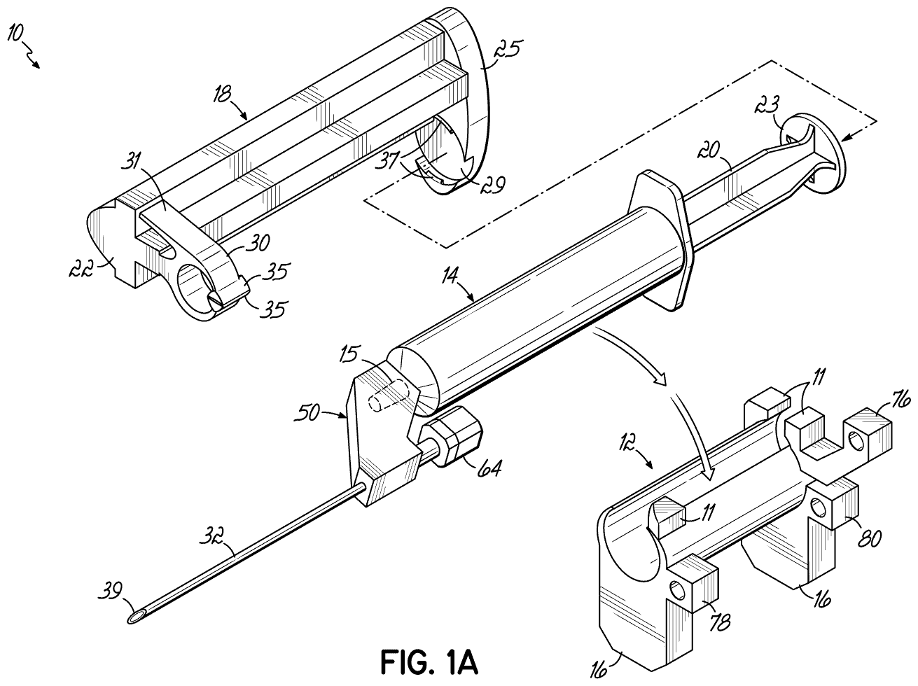

FIG. 1 illustrates an embodiment of a device 10 for advancing a wire into a body space or workspace, such as the vasculature of an animal, such as a human. Such wire advancement can be used for a number of different medical procedures and is generally referred to as the Seldinger technique. Herein, the term vasculature will often be used to generically refer to a space or workspace in tissue in a body, but the use of the invention is not limited to just wire placement in vasculature. Furthermore, while the discussed embodiments are for placement of a guide wire to position other elements in the body workspace, other wire structures may be inserted with the device, and so the element is referred to herein as a “wire”.

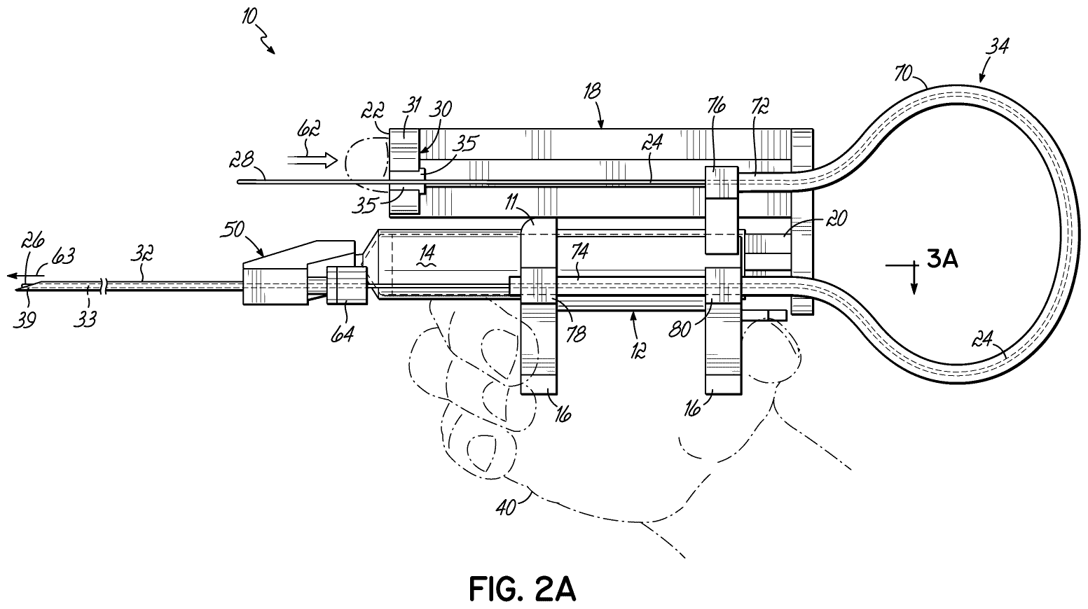

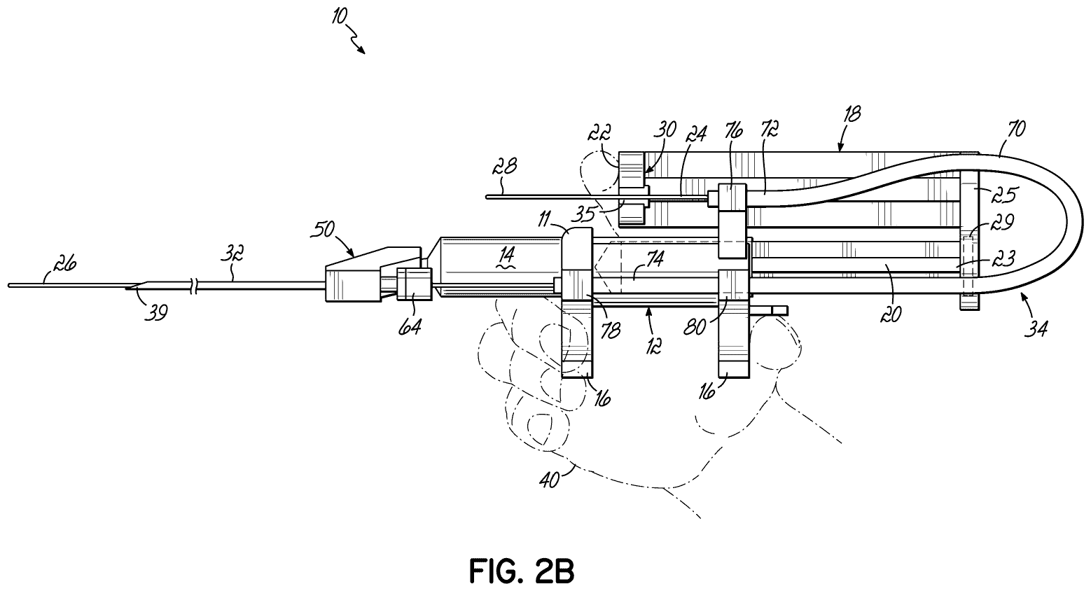

Device 10 includes a housing 12 that includes or is configured for coupling with a syringe body 14. The housing defines a portion, such as one or more grip portions 16 for engagement by a user. The housing provides a way of gripping the syringe body. A slide element or slide 18 is positioned along the outside of the housing and is configured for coupling with a syringe plunger 20. The syringe plunger 20 moves inside the syringe body 14 between a forward position as illustrated in FIG. 2A, for example, and a retracted position as illustrated in FIG. 2B. The slide 18 also includes a grip portion 22 for engagement by a user to move the slide 18 and the syringe plunger 20 with respect to the housing 12 and syringe body 14. The slide acts as an extension of the plunger. Specifically, the slide 18 provides a way for a user to more easily engage and move the syringe plunger 20 from the forward position to the retracted position during the use of the device 10.

Device 10 manipulates and includes a wire 24 for delivery and placement into a tissue workspace. The wire 24 includes a proximal end 26 and a distal end 28. The slide is releasably coupled with a distal end of the wire for moving the distal end and thereby advancing the proximal end forwardly in the needle when the slide moves the plunger from a forward to a retracted position. The slide 18 in one embodiment as shown in FIG. 1 includes a wire gripping structure 30 that releasably engages or grips the distal end 28 of the wire for facilitating movement of the wire with the plunger as described herein. A needle 32 is coupled with the syringe body 14 and is also configured to have a bore for guiding the proximal end 26 of the wire into vasculature or another workspace in accordance with features of the invention. More specifically, movement of the slide 18 and coupled syringe plunger 20 rearwardly to a retracted position away from the needle 32 in the syringe body will draw negative pressure in the needle. The movement of the slide 18 that releasably grips the wire will also seek to advance the wire 24 through needle 32 into the vasculature. To that end, a wire advancing mechanism 34 is coupled generally between the slide 18 and housing 12 as seen in FIG. 1. The wire advancing mechanism 34 acts on the wire to translate movement in one direction into movement in another direction. The mechanism 34 is configured for translating the movement of the wire distal end into movement for advancing the proximal end 26 of the wire forwardly into and through needle 32 when the slide 18 moves the syringe plunger 20 from the forward position to a rearward or retracted position as illustrated in FIG. 2B.

The purpose of the wire advancing mechanism is to reverse or translate the rearward movement of the slide 18 and the accompanying movement of the syringe plunger 20 to the retracted position and create a forward movement of the proximal end of the wire 24 into and through the needle in one movement stroke. As illustrated in FIGS. 2A and 2B, a hand 40 of a human operator engages the respective grip portions 16 and 22 of the housing 12 and slide 18 and moves the slide toward a retracted position along the housing as illustrated in FIGS. 2A and 2B. The wire gripping structure 30 of the slide 18 in FIG. 1 releasably grips the distal end 28 of the wire so the slide and wire are coupled together. The movement of the slide 18 and syringe plunger 20 to a retracted position causes the distal end of the wire to move toward the wire advancing mechanism 34. The wire advancing mechanism 34 reverses or translates that rearward movement of the wire distal end into a forward or advancing movement of the wire proximal end 26 into and through the needle 32. As the slide 18 and plunger 20 are moved further to the retracted position, the proximal end 26 of the wire is advanced further forwardly through the needle, and ultimately into vasculature or another workspace into which the needle is inserted.

In that way, the device 10 of the present invention provides a single motion and unique and smooth forward movement of the wire through needle 32 and into the vasculature as the plunger is moved to the retracted position and a negative pressure is drawn and released with respect to the syringe plunger 20 moved by slide 18. The slide 18 is coupled with both the wire and plunger and moves both the syringe plunger 20 as well as wire 24 in a single stroke or movement of the slide 18 as described further herein.

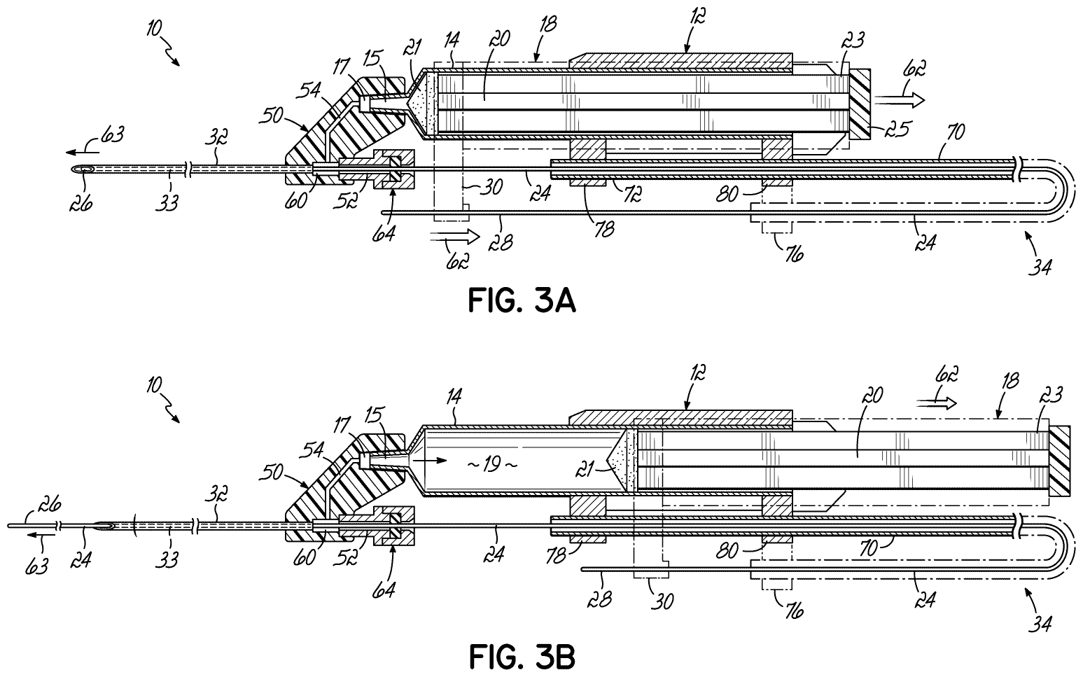

The needle 32 is configured for coupling with the syringe body and has at least one base or lumen for guiding a proximal end of the wire. Device 10 further includes a needle mount 50 for coupling the needle 32 with the device. The needle mount 50 couples the needle 32 with both the syringe body 14 as well as the wire 24. FIGS. 3A and 3B illustrate cross-sectional top views of the device with the slide and syringe plunger in the forward position and then the slide and syringe plunger in the retracted position used for advancing wire 24. The needle mount 50 includes a guide passage 52 coupled with needle bore 33 for directing movement of the wire 24 into and through the needle 32. The needle mount 50 also includes a vacuum passage 54 that is coupled with the syringe body 14 to create a negative pressure in the needle bore when the syringe plunger is moved to the retracted position as illustrated in FIG. 3B. As described herein, movement of the slide and the syringe plunger to the retracted position as shown in FIG. 3B will draw a negative pressure within the needle 32 through needle mount 50. As long as the needle is blocked by tissue, that negative pressure will generally prevent significant rearward movement of the slide and syringe plunger to the retracted position under the force of a gripping human hand on the respective grip portions 16 and 22 of the housing and slide. The slide and plunger may move slightly to create the negative pressure with hand pressure but will be prevented from moving readily to the full retracted position. Once the needle tip penetrates through the tissue and the vasculature and the negative pressure is broken, the slide and syringe plunger may be drawn or moved more easily in the device 10 to the retracted position. Simultaneously, the movement of the slide and plunger also moves the wire gripping structure 30 rearwardly with respect to needle 32. This pushes wire 24 through the wire advancing mechanism 34 and turns or reverses or translates the wire movement and then advances the proximal end 26 of the wire forwardly through needle 32 as illustrated in FIG. 3B.

In the embodiment of the invention illustrated in FIGS. 1-3B, the needle mount 50 creates an airtight passage in both the vacuum passage 54 as well as the guide passage 52. The needle 32 is hollow and includes the bore 33 that is dimensioned to allow for passage of wire 24 therethrough. The bore 33 is also dimensioned to allow the simultaneous passage of air and/or fluid through the needle 32 when the wire 24 moves within the needle. The needle mount 50 forms an intersection point 60 for the vacuum passage 54, the bore 33 of the needle, and the guide passage 52 for the wire 24. The syringe body 14 forms an airtight seal with needle mount 50 and passage 54. In one embodiment of the invention as illustrated in the figures, the syringe body 14 includes a tip 15 that fits in an appropriately formed opening or cavity 17 in the needle mount. The vacuum passage 54 through engagement with the intersection section 60 and needle bore 33 provides a connection between the needle 32 and the internal space 19 of the syringe body as illustrated in FIG. 3B. When the syringe plunger 20 is forced into the retracted position by a user as illustrated by arrows 62, pressure is created to draw gas or fluid in through needle 32, through the vacuum passage 54 and into the internal space 19 of the syringe body. In order to ensure that such a negative pressure is created in the needle 32 and particularly in the bore 33 of the needle, the passage 52 which guides wire 24 also has to be airtight in the embodiment of FIG. 1. To that end, the needle mount includes a sealing mechanism 64 that engages with guide passage 52 and wire 24 for providing the airtight seal in the guide passage. That is, the sealing mechanism 64 engages with wire 24 as it passes through the sealing mechanism and into the guide passage 52 so that negative pressure is maintained and wire 24 may move through the guide passage of the needle mount and through needle 32 at the same time that negative pressure is being drawn through movement of the plunger 20 within the syringe body 14. In one embodiment, the sealing mechanism 64 may entail a rubber or silicone element having a hole for passing the wire. The wire 24 could be a smooth wire or a coiled wire. A coiled wire may have a coating thereon or could have a substance applied, such as petroleum jelly, as a coating material.

In the present invention, using a needle mount 50 as shown separates the fluid/air passage of the syringe from the wire passage. This allows the syringe to be removed while the wire is still in the needle and provides the ability to perform a Fabian test while the wire is already positioned or advanced. This can verify that the wire is not in an artery rather than an intended vein.

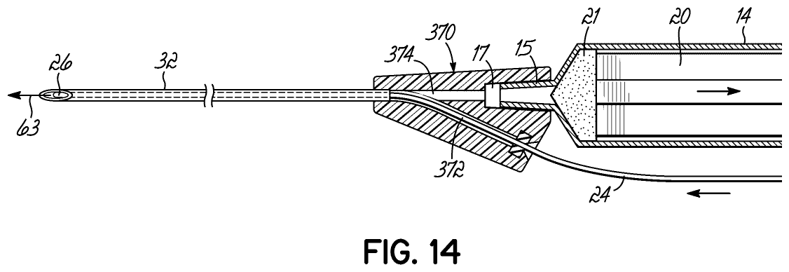

The needle mount 50 has a syringe passage that is adjacent to but not inline with the needle. Rather the guide passage is inline with the needle to move the wire straight into the needle. This reduces friction and provides easier advancement. In another embodiment as shown in FIG. 14, the syringe or vacuum passage is aligned with the needle and the wire enters at an angle. A pre-manufactured needle may be used in the needle base. Alternatively, the needle may be manufactured directly with the needle base. In one embodiment, the needle base may be made of a clear material or include a clear area to more readily see when fluid enters the needle.

As may be appreciated, the syringe plunger 20 may be configured to resemble a typical syringe plunger having an elongated body portion which is terminated in a rubber or plastic stopper 21 that seals with the internal wall of the syringe body 14 and creates an air and fluid tight seal. A force provided in the direction of arrow 62 on the end 23 of the syringe plunger 20 will move the syringe plunger 20 and stopper 21 to a retracted position as illustrated in FIG. 3B. FIG. 3A shows the syringe plunger 20 and stopper 21 in the forward position against the end of the springe body 14 and proximate the tip 15. If the needle tip is blocked, negative pressure is created in the syringe.

In accordance with one aspect of the invention, the housing 12 is configured for coupling with the syringe body. In one embodiment, the syringe body 14 may be formed as a unitary structure with the housing wherein the syringe body includes the integrally molded housing and grip portions. In another embodiment of the invention, the housing 14 may be configured to receive a separate syringe body 14 with the syringe body in a proper position with respect to the grip portions 16 of the housing. Similarly, the slide 18 may be formed and configured for coupling with a syringe plunger or the slide may be formed together with or as an extension of a syringe plunger as a single piece, such that the slide and grip portions move as one piece with the syringe plunger. Alternatively, the slide 18 may be formed and configured to couple to a separate syringe plunger in order to couple the movement of one to the other. In that way, a housing 12 and slide 18 might be configured and dimensioned to receive and utilize a typical syringe having a syringe body and syringe plunger.

To that end, FIG. 1A shows one embodiment of the device 10 wherein housing 12 includes a generally cylindrical passage 17 configured for receiving syringe body 14 to couple with the syringe body. The slide 18 is configured to slide on or adjacent to the housing 12 and is positioned generally parallel to the housing. Guide elements 11 on housing 12 may engage the slide 18 to ensure a straight movement of the slide to the retracted position with respect to the housing and the syringe body. Slide 18 has an end 25 that has an indent 29 or other structure for engaging an end 23 of a syringe plunger 20 to hold the end 23 and couple the slide and plunger. The plunger and slide engagement may be a friction fit or the plunger end 23 might be held in the indent by one or more tabs 37 that snap on the end 23. In that way, the slide acts as an extension of the plunger to move the plunger.

The housing 12, syringe body 14, slide 18, and syringe plunger 20 may be formed of a suitable material, such as a plastic material that is lightweight and may be sterilized. The material provides sufficient rigidity for movement of the plunger in the syringe body as well as gripping and guiding of the wire 24.

In accordance with one feature of the invention, the wire advancing mechanism 34 is configured for advancing the proximal end of the wire forwardly into and through the needle when the slide moves the syringe plunger from the forward position to the retracted position. That is, the wire advancing mechanism 34 is functional to translate or change the direction of the wire such that moving the syringe plunger rearwardly to a retracted position away from the needle actually simultaneously advances the wire forwardly into and through the needle and ultimately into vasculature into which the needle penetrates as described herein. This proceeds in one single and fluid motion wherein moving the plunger to the retracted position simultaneously advances the wire forwardly into the needle.

FIGS. 1-3B illustrate one wire advancing mechanism 34 in accordance with an embodiment of the invention that includes a loop guide 70 for reversing or translating the direction of the wire movement. The loop guide is generally positioned between the slide 18 and housing 12 so as to provide the reversal or translation in the direction of the wire movement with respect to the movement of the slide 18 and the syringe plunger. Specifically, referring to FIG. 1, the wire advancing mechanism 34 includes a loop guide 70 that has an input end or distal end 72 positioned to receive a wire from the wire gripping structure 30 of the slide 18 and an output end or proximal end 74 for guiding a proximal end of the wire 24 and outputting the wire into needle mount 50 and ultimately into needle 32. The loop guide 70 may be a closed tube as illustrated in FIG. 1. Alternatively, the loop guide might be open and form an appropriate structure for capturing wire 24 and guiding it from the distal end 72 to the proximal end 74. As illustrated in FIG. 1, the housing 12 may be appropriately formed to include mount sections 76, 78 that interface with and contain the respective distal end 72 and proximal end 74 of the loop guide 70. Depending upon the length of the housing, as well as the length of the loop guide 70 and the length of the syringe body, slide, and syringe plunger, one or more additional mount sections 80 may be implemented in the housing 12 for holding a portion of the loop guide 70 between the distal end 72 and proximal end 74. As illustrated in FIG. 2A, the wire 24 is contained by loop guide 70 which reverses direction of the movement of the wire 24 and slide 18 as illustrated by reference arrow 62 and changes the direction of movement of the proximal end 26 of the wire 24 in the direction of arrow 63. In that way, negative pressure or a vacuum may be drawn through syringe body 14 by movement of slide 18 and the plunger rearwardly in the direction of arrow 62 and simultaneously, when the pressure is released, the proximal end 26 of the wire will advance forwardly in the direction of arrow 63 or in the direction of the movement of needle 26 into the workspace as described herein.

The terms proximal and distal as used herein, particularly with respect to the wire 24 and its ends or sections are used to indicate one end proximate to the body which proceeds through the needle and into the workspace and the other end which is spaced from the needle and is gripped and pushed by movement of the slide 18 and the plunger. It generally refers to sections or portions of the wire relative to each other, rather than referring specifically to a tip or terminating end of the wire. Accordingly, the proximal end 26 of wire 24 refers to that portion or section which moves into and/or through the needle while the distal end 28 of the wire refers to that portion or section of the wire which is releasably coupled with the slide 18 and moved by the slide to push the proximal end of the wire into position in the tissue workspace.

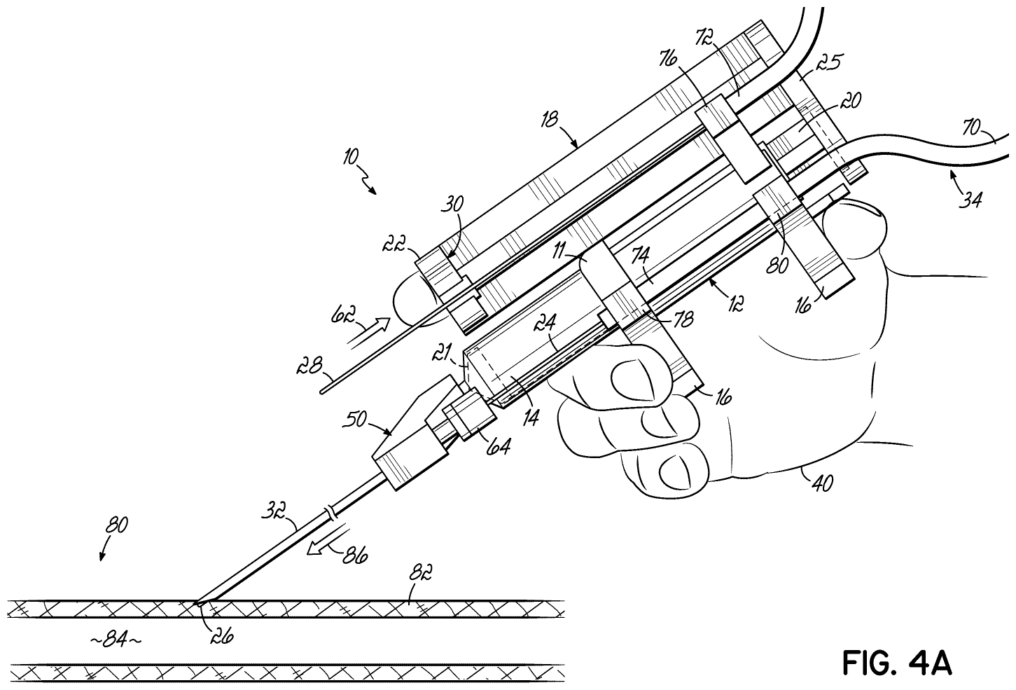

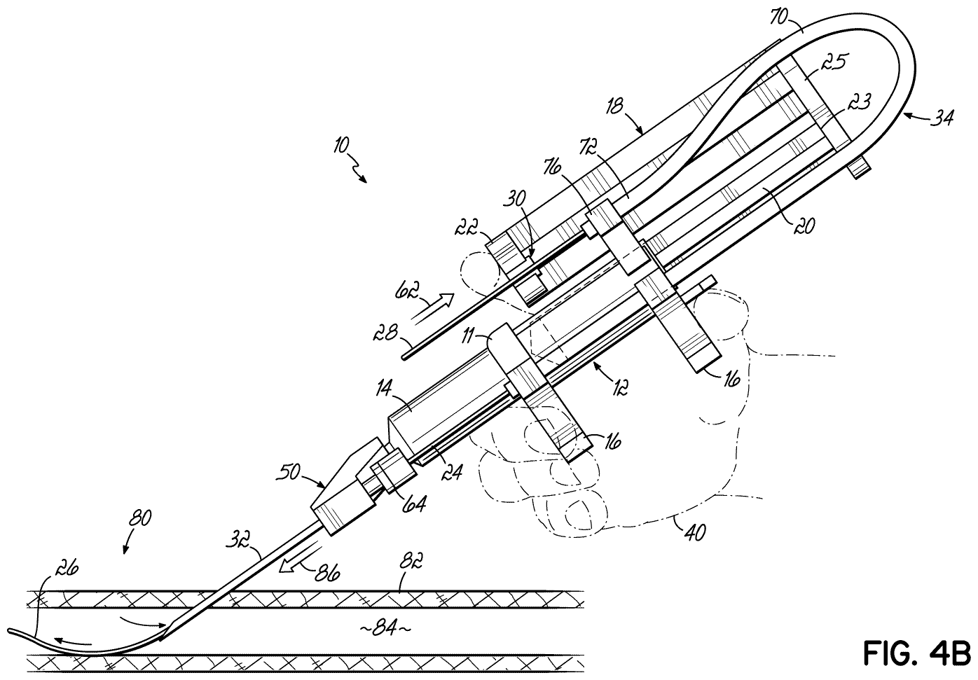

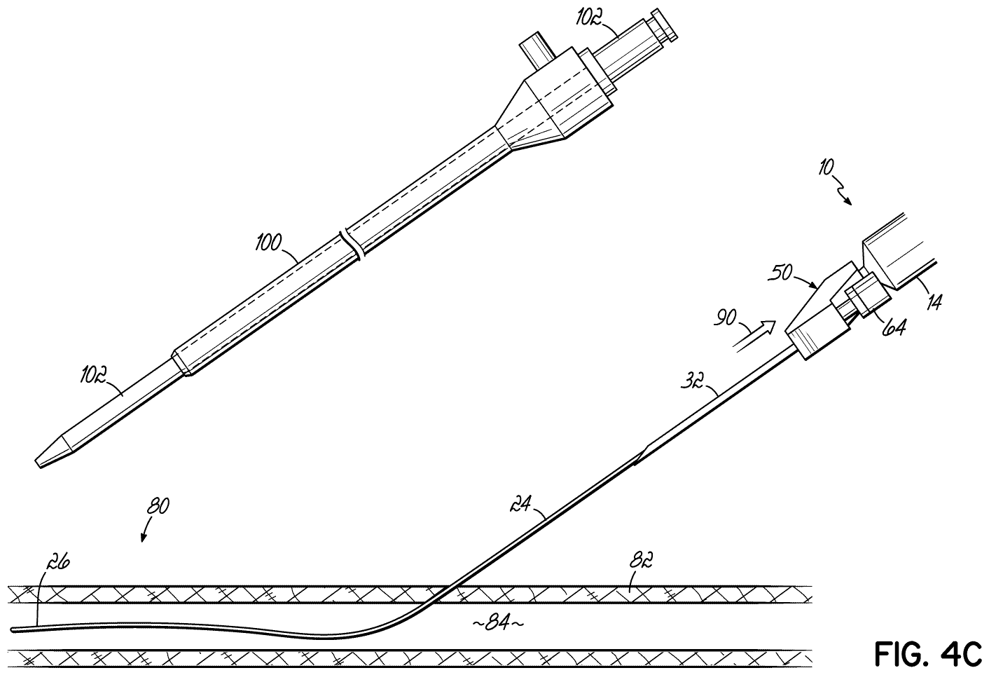

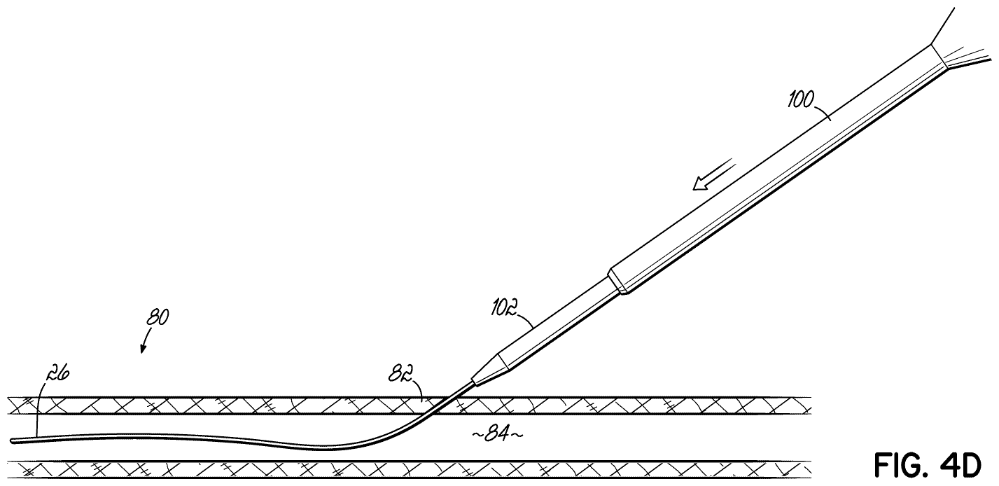

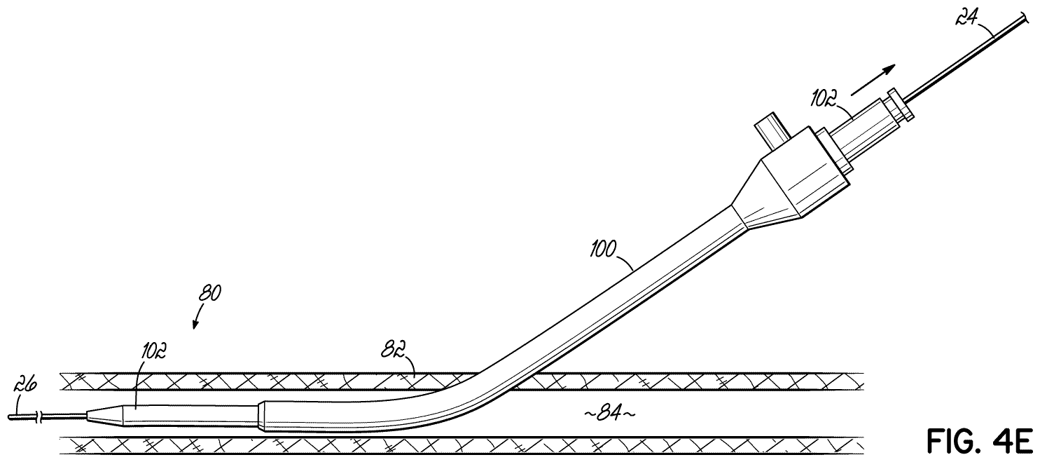

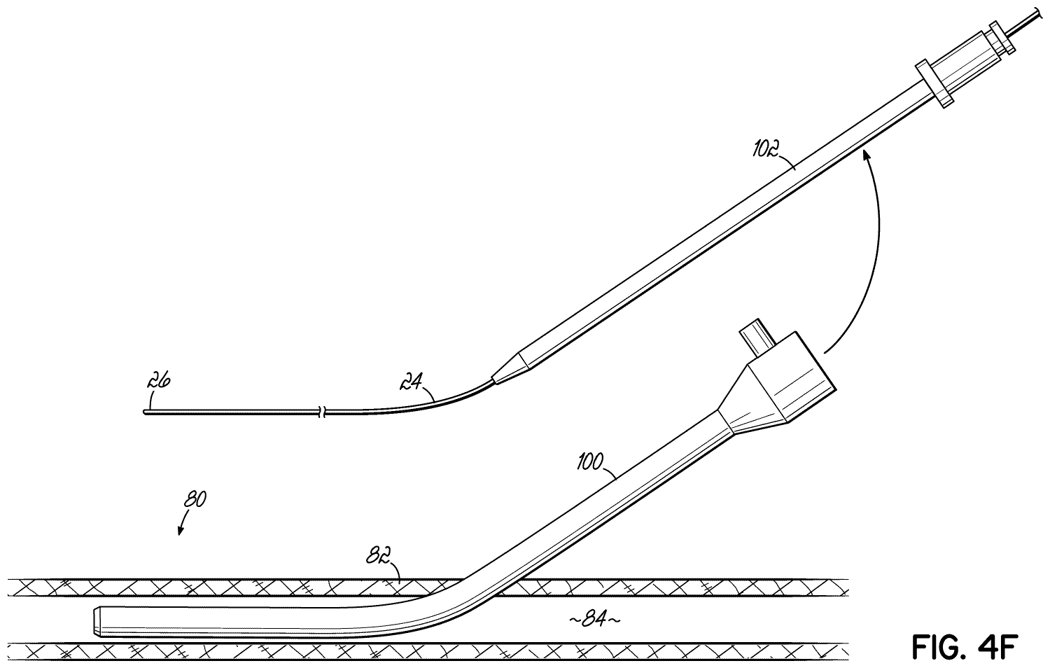

FIGS. 4A-4F illustrate use of the present invention and an inventive method for performing a Seldinger technique or other technique for positioning a wire and other elements, such as a catheter, into the vasculature of a patient or some other cavity or workspace. Device 10 is gripped by the hand 40 of a user for engaging the syringe. Specifically, the fingers of the hand are positioned at the various grip portions 16 and 22 of elements coupled with or part of the syringe. Generally, the grip portion 16 of the housing are held by the hand 40 and fingers of a user while the index finger or another finger is positioned proximate the grip portion 22 of the slide 18 for moving the slide with respect to the housing. In that way, the syringe body and plunger are engaged for movement.

FIG. 4A shows vasculature of an animal, such as a human that includes one or more layers of skin or tissue 82, which confine or define an internal passage (workspace) 84 that carries the blood of the patient. Device 10 is gripped and the needle 32 is positioned against the skin 82 and is pushed in the direction of arrow 86 into the skin and subcutaneous tissue reflected by reference element 82. The tip 33 of the needle 32 and its bore are blocked initially by the skin and subcutaneous tissue 82. A retraction force in the direction of arrow 62 on grip portion 22 by the finger of a user's hand will move the plunger and create and draw a negative pressure within the syringe body 14 because the needle tip is blocked. Typically, the movement as shown of slide 18 will seek to move plunger 20 from the forward position to the retracted position and thus seek to draw air or fluid into needle 32. However, since the needle tip 33 is within the subcutaneous tissue or skin 82, no air or fluid can enter the needle 32. Therefore, a negative pressure or vacuum is created within the syringe body 14 and particularly within internal space 19 of the syringe body as the user seeks to move slide 18 and draw in fluid/air. Generally, the wire of 24 will be threaded into needle 32, and may be threaded a distance sufficient to be proximate to the tip 33 at the start of the process. As shown in FIG. 4A, the proximal end of the wire may be right at the tip 33 or rearwardly of the tip as it is passed into the tissue 82. The slide 18 may be slightly movable in the direction of arrow 62 before the plunger meets significant resistance from the vacuum drawn within the syringe body 14. Since movement of the plunger 18 and the associated wire gripping structure 30 will move the wire 24 slightly, generally it is desirable to position the tip of the wire proximal end 26 or the terminal end of the wire to be slightly back from the tip 33 of the needle at the beginning of the procedure so that it will not pass out of the needle at the short initial drawing of the plunger and vacuum. The distance may be determined by the size of the syringe body/plunger and can be adjusted by a user as desired by moving the wire manually within the gripping structure 30 so that the device 10 may be customized to a desired amount of negative pressure and slide movement before the distal end of wire moves out of the tip of the needle 33. In some embodiments, the wire might be 1-2 centimeters back from the tip. The space 84, such as a workspace within vasculature defined by tissue 82 is the ultimate target for the tip of the needle 33 as well as the wire 24.

Referring to FIG. 4B, once needle 32 is passed through the tissue 82 and into space 84 fluid/air may be drawn in and there is no longer negative pressure or a vacuum. Therefore, the slide 18 and syringe plunger 20 may be moved more easily in the direction of arrow 62. That is, with the needle tip 33 in space 84 and without the vacuum, the slide and syringe plunger may be drawn further back to the retracted position as illustrated in FIG. 4B. With that movement, the distal end 28 of the wire 24 is moved toward the wire advancing mechanism 34. That is, with the loop guide 70 embodiment illustrated in FIGS. 4A-4F, the wire is moved toward and through the loop guide 70 and the direction is reversed to move the wire proximal end 26 in the direction of the needle, reflected by arrow 86 as shown in FIG. 4B. That is, in accordance with one aspect of the invention, the wire advancing mechanism is configured for translating or reversing wire movement and advancing the proximal end 26 of the wire forwardly into and through the needle when the slide 18 moves the syringe plunger 20 from the forward position to the retracted position. As will be appreciated, the retracted position may be any position in the syringe body rearwardly of the most forward position wherein a vacuum is drawn. Therefore, the retracted position is generally relative to the forward position rather than being an absolute rearward position of the syringe plunger in the syringe body. A user controls the retraction of the slide and plunger. As the distal end 28 of the wire is moved rearwardly with the slide 18, the proximal end of the wire 24 is advanced through needle 32 and into the target space 84. As noted, while the example set forth herein discusses movement of the wire into vasculature, the target space may be any suitable vessel, abscess, or body cavity or other target of a patient and thus using the device 10 is not limited to a specific vasculature, although vasculature is used herein as a term to generally note the target space of the wire in the patient.

Once the wire 24 has been entered into the target space, the distal end 28 of the wire may be removed from the wire gripping structure 30. To that end, the gripping structure 30 might include lever arm 31 as shown in FIG. 1 which may be compressed in order to open up the wire gripping structure 30 to release the distal end 20 of wire 24. Thereafter, the wire 24 is free to be manipulated by a user rather than being coupled to slide 18 and the plunger. Since the wire 24 is already well within the target space 84, small, unintentional movements of the wire by a user will not remove it from the target space 84. Referring to FIG. 4B, the wire 24 is generally grippable at the exposed sections reflective of the distal end 28 as well as the portion of the wire proximate housing 12 that is rearward of the proximal end 26. A user can grip the wire at either of the exposed locations for further manipulation. For example, while the device 10 is held by one hand, the other hand of a user could be used to advance the wire further. Once the wire has been advanced to a desirable distance within space 84, it can be used for inserting other elements into the target space 84. For example, the wire can be used to advance a catheter with a dilator into the space.

Referring to FIG. 4C, device 10, and particularly the needle 32 and needle mount 50, may be removed from the wire. However, it may be desirable initially to only remove the syringe body and the rest of the device from the needle mount and leave the needle in place for additional procedures. For example, a Fabian test can be performed by hooking up tubing to the opening in the needle mount coupled with vacuum passage 54, or to the passage initially coupled with the syringe body 14. For procedures such as the positioning of a catheter 100, the device 10, including the needle 32 and needle mount 50 are removed and pulled up the wire as shown in FIG. 4C. Specifically device 10 and needle 32 and needle mount 50 can be removed in the direction of arrow 90 from the wire 24 positioned in space 84. The wire 24, in one use, may be for the purpose of guiding and positioning a catheter into the target space 84, such that wire 24 is used for guidance. Referring again to FIG. 4C, a catheter 100 around a dilator 102 to be threaded onto the distal end 28 of the wire once device 10 and needle 32 have been removed.

Referring to FIG. 4D, once the wire 24 has been threaded into the dilator 102 and catheter 100, those elements may be slid through the opening in tissue 82 and the same opening utilized to insert the wire. As shown in FIG. 4E, dilator 102 and catheter 100 can then be positioned in space 84 for further procedures. Then, wire 24 and/or dilator 102 may be removed from the catheter 100 and space 84 as illustrated in FIG. 4F. Wire 24 may be removed first and then the dilator 102 or they could be removed simultaneously to leave the catheter 100 in position for the performance of further procedures within the target space 84, such as vasculature.

The device 10 provides significant advantages over techniques for introducing and advancing a wire and other elements within a target space of a patient. Device 10 directly couples the drawback of a syringe plunger to the advancement of wire 24 via the releasable wire gripping structure of 30. The same force that is used to move the slide and plunger is generally the same force used to move the wire into the needle and the workspace. That is, there are no intervening mechanisms in device 10 so that the wire movement haptic feedback is directly to the plunger and slide and a user's fingers. The wire may then be advanced once the needle tip enters any target vessel, abscess, body cavity, or other space. With a single hand, a user can position the wire within the target space. The function of the syringe body 14 and the movement of the syringe plunger therein through slide 18 and the wire movement through loop 70 is the only resistance encountered by a user and thus allows for greater haptic feedback in the process. The direct coupling between movement of the syringe plunger and simultaneous advancement of the wire allows a user to have a better feel of how the wire is advancing and provides faster feedback that the wire may not be advancing appropriately. For example, if the wire runs into the wall of a vessel or an occlusion or if the needle is withdrawn from the target space prior to wire advancement, a user has immediate feedback via the housing and slide and the grip portions 16 and 22 provided on the housing 12 and slide 18. Furthermore, the wire 24 advances external to the syringe housing 12 or syringe plunger as illustrated in FIG. 1. As discussed further herein, this allows for other elements to be preloaded onto the wire for placement using the wire in target space 84.

The present device 10 and other alternative embodiments of the invention as described herein, can be held in one hand for use to provide a generally single movement. In an alternative embodiment, engagement by a robot rather than a hand might be used. The wire can be advanced with the same hand or movement that pulls the syringe plunger. A user is controlling the advancement of the wire and no spring, lever intervening, or other mechanism removes that control from a user. If the wire meets resistance as felt in the device, a user can stop moving the plunger and advancing the wire. Furthermore, an additional advantage is that the device 10 and its related embodiments allow for a longer wire to be used. The wire advancing mechanism 34, particularly the loop guide 70 or other guide structure, can be configured and dimensioned based upon the desired length of the wire. The additional wire can then allow catheters/sheaths and dilators to be preloaded onto the wire for introduction into the target space over the wire as discussed further herein. In accordance with another feature of the invention, different sizes of syringe bodies 14 may be used with different amounts of fluid from the target space correlated to a greater or lesser movement of the wire based upon movement of the slide and wire gripping structure 30. That is, if a syringe body 14 and syringe plunger having a larger diameter are used, more air/fluid is needed to translate the slide rearwardly and move the syringe plunger to the retracted position at a certain distance. If it is preferable to have a more minimal amount of air/fluid through the syringe to advance the wire a desired distance, a smaller diameter syringe body 14 and syringe plunger 20 may be used. The wire and syringe decoupling, so that the wire can be advanced independently of fluid draw, and the ability to adapt the fluid draw is useful if a target space is small. If significant fluid draw may collapse a target space, this ability to move the wire without too much draw or independently of further draw is advantageous.

In the present invention, with the single hand of a user, the entry of the target space can be determined and the slide 18 and syringe plunger 20 moved rearwardly to a retracted position while the wire is advanced forward simultaneously and generally in a single stroke. Furthermore, the force that is used to advance the wire is created directly by a user and thus can be more sensitively and accurately applied as opposed to another mechanism, such as a spring or lever that advances the wire. That is, there is generally a 1:1 ratio of force to draw the plunger and advance the wire using device 10. This provides for very sensitive feedback in use of the device and movement of the wire to the user through the user's hand.

The present device 10 and other disclosed embodiments can be used with a range of needle sizes, syringe body sizes, and wire sizes. A tapered wire may be used for the wire element to allow fluid in the needle to easily progress past the wire into the syringe body. Furthermore, a wire with a coiled wire tip might be utilized while the rest of the wire is smooth. In that way, the seal around the wire could be airtight, but the fluid/air could more easily go around the tip of the wire into the needle. As noted, the housing 12 and syringe body 14 may be formed such that they are a unitary piece. Similarly, the slide 18 and syringe plunger 20 may be formed together as a unitary piece. Then the syringe plunger with grip portions and a wire gripping structure would engage the syringe body with its own grip portions to provide the desired interaction of the components. Alternatively, the housing 12 and slide 18 may be configured separately from the syringe components. In such a case, syringe body 14 would fit or snap or otherwise couple with the appropriately constructed housing 12 while the syringe plunger 20 would fit into or snap or otherwise couple appropriately with the slide where the slide 18 and syringe plunger interact as disclosed.

The wire gripping structure 30 as illustrated in FIGS. 1 and 2A may be a clamp type structure with opposing clamp ends 33, 35 biased together to frictionally grip or clamp wire 24 at its distal end 28. The lever structure 31 as shown in FIG. 1 may be operated to open and close the clamp ends 33, 35 for gripping or releasing the wire 24. The pair of clamp ends may be utilized to releasably grip the distal end of the wire. The grips should be tight enough to provide the necessary movement of the wire upon moving the slide 18 and the plunger to a retracted position under the force of a user's hand. Other wire gripping or wire clasping structures may also be used as long as the wire is releasable from the plunger/slide movement.

The present invention provides an ergonomic advantage and stability utilizing a single hand of a user. The grip portions 16 that extend from housing 12 allow for the thumb and one or more of the middle fingers to hold the device and stabilize the syringe body for example. The grip portion 22 positioned at the forward end of the slide 18 allows the index finger on the same hand to easily and comfortably move the plunger and draw negative pressure and then subsequently advance the wire upon the needle entering the target space. The thumb and one or more middle fingers can firmly support the device 10 and syringe body while the index finger can separately control the motion of the slide, syringe plunger, and the wire. This allows a user to keep the needle tip very steady to prevent removal of the needle from the target space while the wire is simultaneously being advanced.

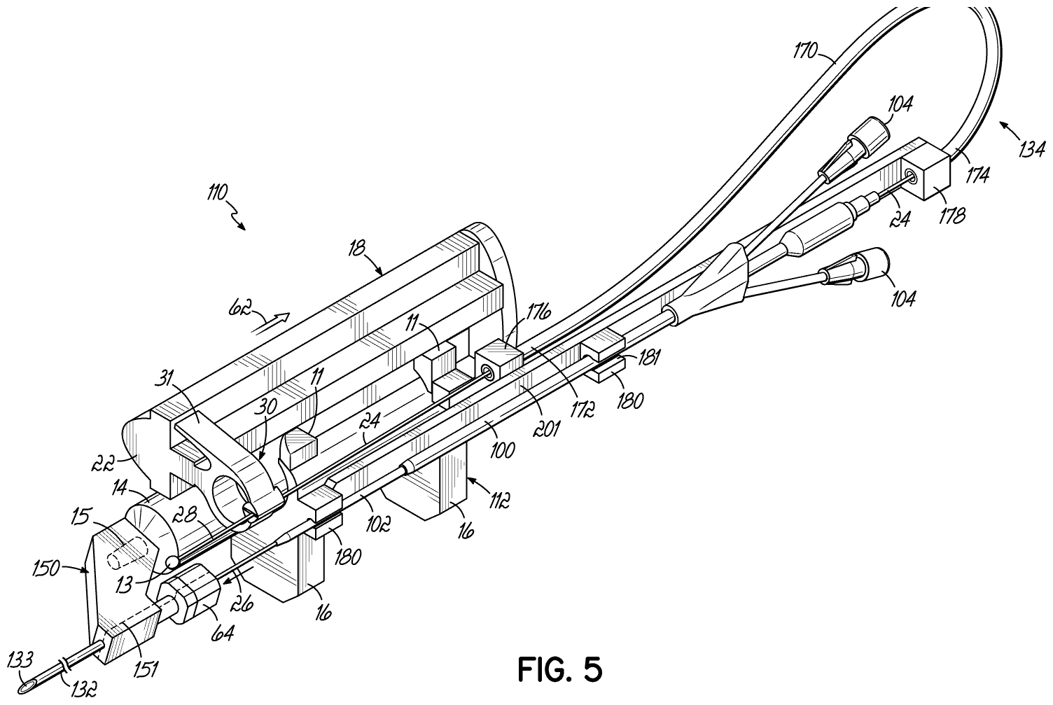

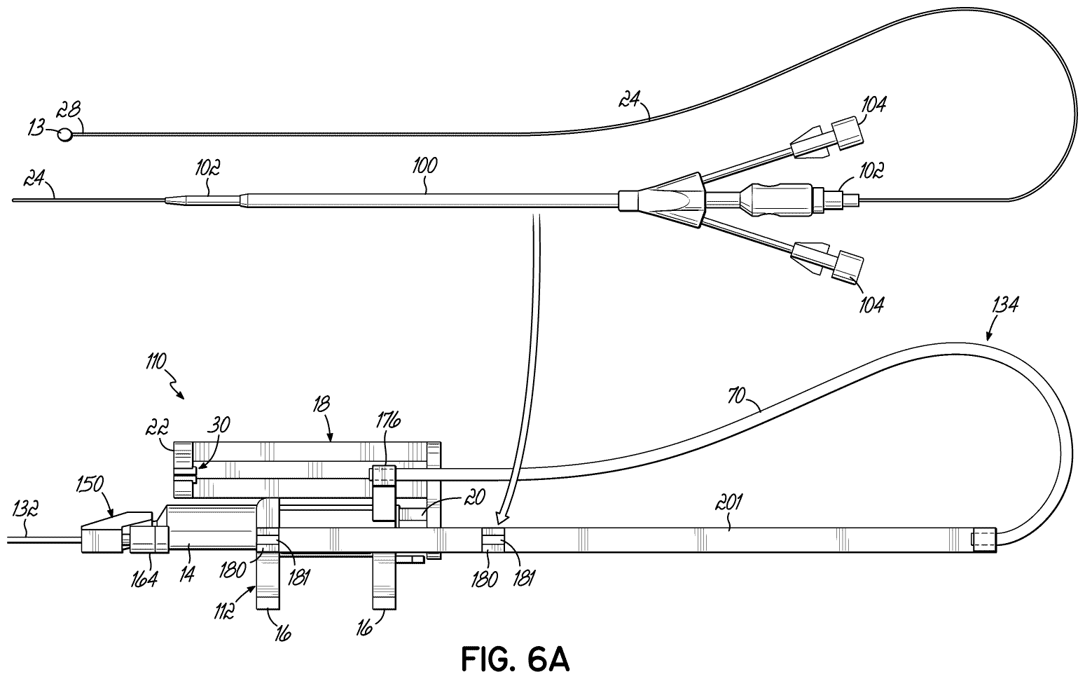

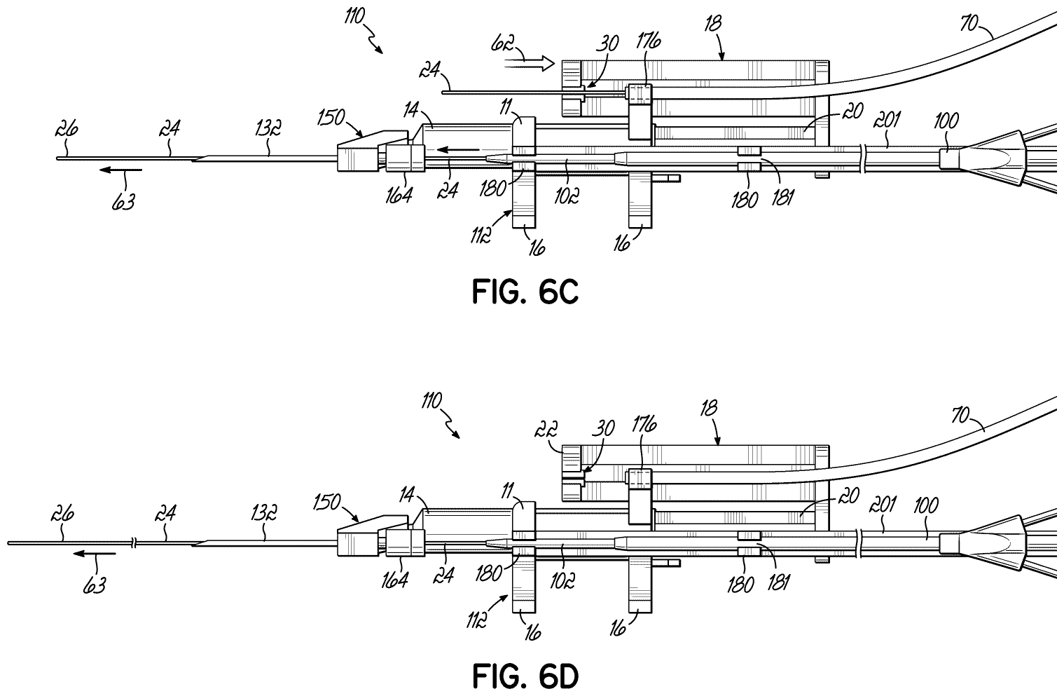

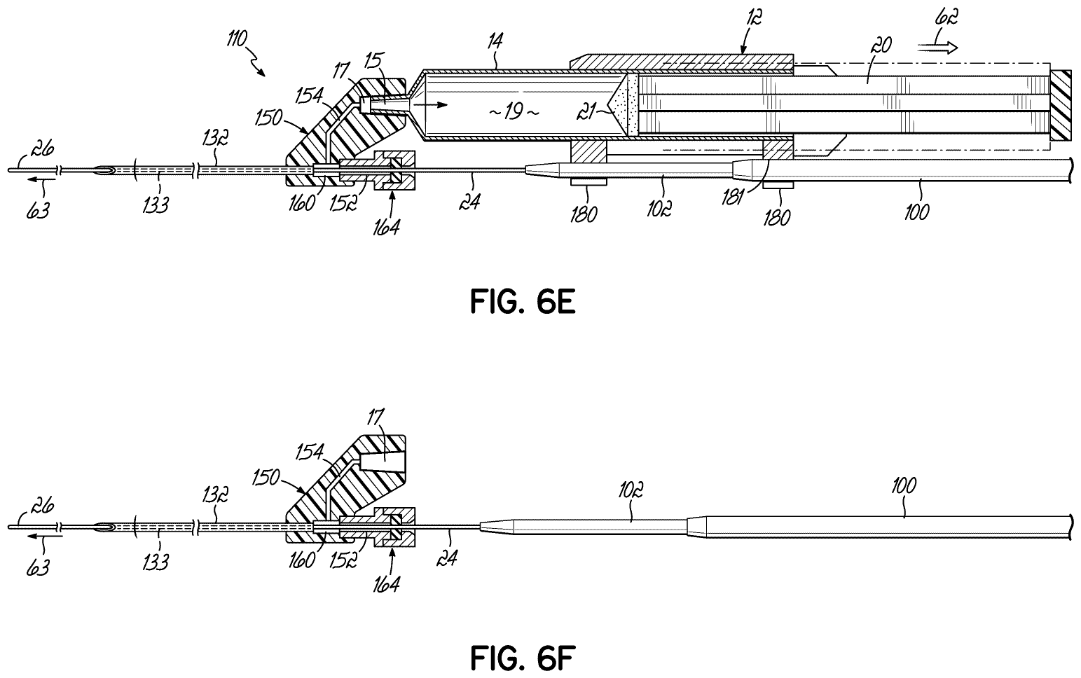

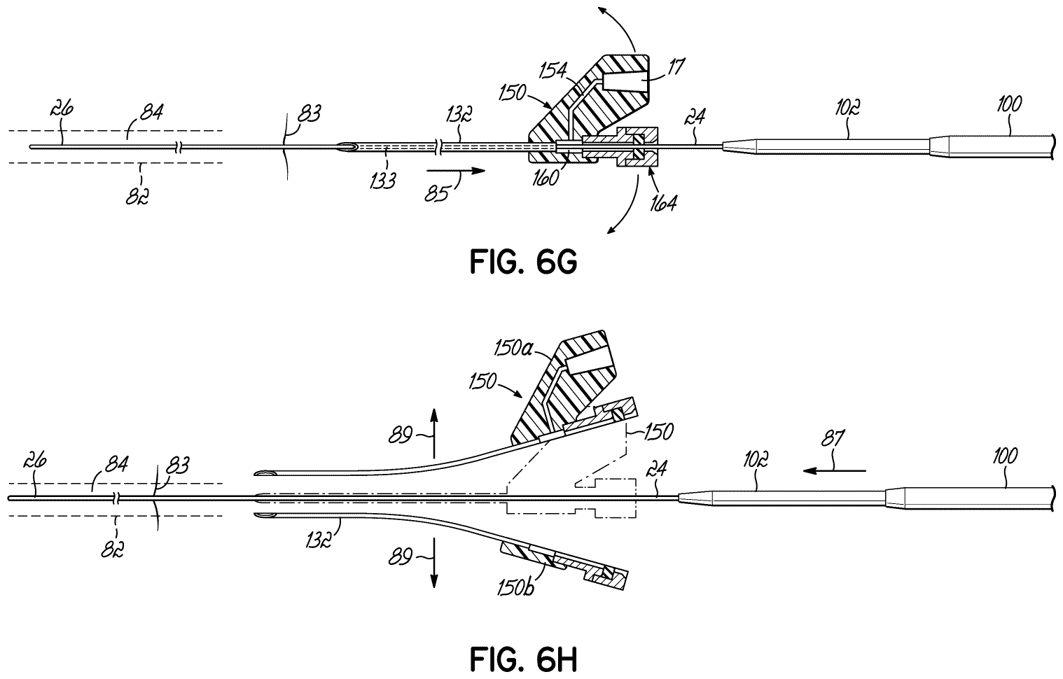

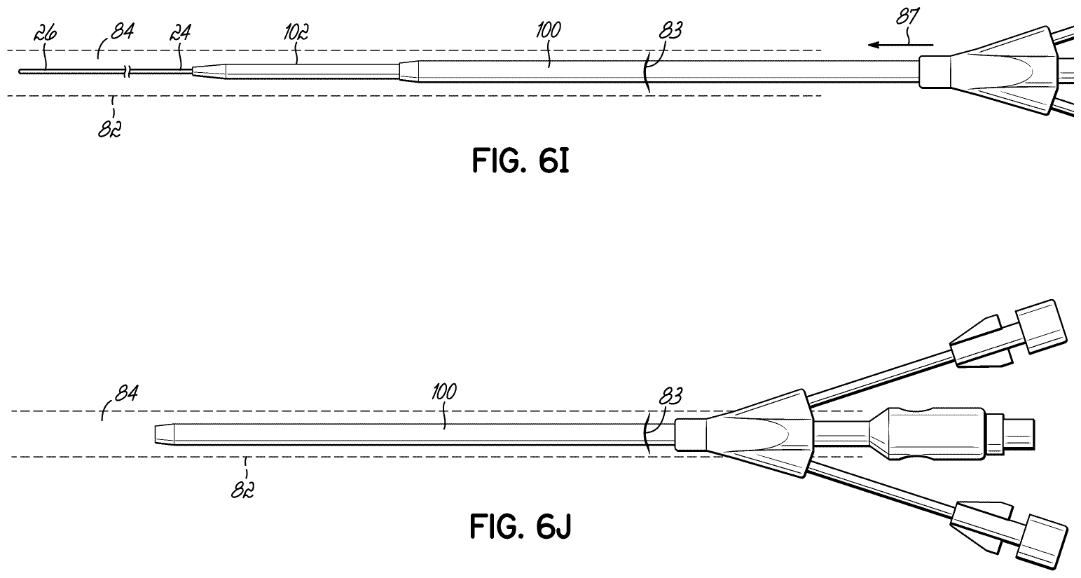

FIG. 5 illustrates an alternative embodiment of the device 110 similar to device 10 but providing for the advancement and positioning of a wire preloaded with a catheter, dilator or other element into a target space in tissue, such as into the vasculature of a human. As with other embodiments of the invention, such wire advancement can be used for a number of different medical procedures. Similar elements between the devices 10 and 110 will share some common reference numerals for common elements. The device 110 includes a housing 112 that includes or is configured for coupling with a syringe body 14 and grip portions 16 for engagement by a user. As with device 10, the housing and grip portions could be part of a unitary syringe body or separate from the body. A slide 18 is positioned adjacent housing 14 and is part of the plunger or configured for coupling with a syringe plunger 20. The syringe plunger 20 moves inside the syringe body 14 between a forward position as illustrated in FIG. 6B, for example, and a retracted position as illustrated in FIG. 6C. The slide 18 also includes a grip portion 22 for engagement by a user to move the slide 18 and the syringe plunger 20 with respect to the housing 112. Specifically, the slide 18 moves the syringe plunger 20 from the forward position to the retracted position during the use of the device 10. As described herein, the housing 112 may be dimensioned to accept a catheter arrangement in front of the wire advancing mechanism.

Device 110 also includes a wire 24. The wire 24 includes a proximal end 26 and a distal end 28. The slide 18 also includes a wire gripping structure 30 that releasably grips the distal end 28 of the wire for facilitating movement of the wire as described herein. In the embodiment of the device 110 illustrated in FIG. 5, the wire 24 is preloaded with other components to be inserted into the target space in the tissue as guided by the wire. Specifically, as illustrated in FIG. 5, a dilator 102 and catheter 100 (i.e., catheter arrangement) are loaded onto the wire. The catheter 100 may include one or more ports 104 that feed into the catheter depending on the medical procedure for which the catheter will be used. A needle 132 is coupled with the syringe body 14 and is also configured for guiding the proximal end 26 of the wire into a workspace, such as a vasculature space, in accordance with features of the invention. Similar to device 10, the movement of the slide 18 and syringe plunger 20 rearwardly away from the needle 132 and the body will draw a negative pressure and will also seek to advance the wire 24 through a bore of needle 132 into the vasculature or other target space in a single movement.

To that end, a wire advancing mechanism 134 is coupled generally between the slide 18 and housing 112 as seen in FIG. 1. The wire advancing mechanism 134 guides the wire and is configured for advancing the proximal end 26 of the wire forwardly into needle 132 when the slide 18 moves the syringe plunger 20 from the forward position to a retracted position as illustrated in FIG. 6C.

The purpose of the wire advancing mechanism is to reverse or translate the rearward movement of the slide 18 and the accompanying movement of the syringe plunger 20 to the retracted position and create a forward movement of the wire 24 into and through the needle 134 in one movement stroke. As described herein, a hand of a human operator or some other mechanism, such as a robot, engages the respective grip portions 16 and 22 of the respective housing and slide and moves the slide 18 rearwardly toward a retracted position along the housing as illustrated in FIGS. 6B and 6C. The wire gripping structure 30 of the slide 18 grips the distal end 28 of the wire. The movement of the slide 18 and syringe plunger 20 to a retracted position causes the distal end 28 of the wires to move through the wire advancing mechanism 134. The wire advancing mechanism reverses or translates that rearward movement of the wire distal end into a forward movement of the wire proximal end 26 into and through the needle 132. As the slide 18 and plunger 20 are moved to the retracted position, the proximal end 26 of the wire is simultaneously advanced through the needle, and ultimately into the target space into which the needle is inserted as described herein.

In the embodiment illustrated in FIGS. 5-6J, the housing 112 is configured to space the wire advancing mechanism 134 further rearwardly of the housing 112. In accordance with the invention, the wire advancing mechanism 134 operates similarly to mechanism 34 and guides the wire and is configured for advancing the proximal end of the wire forwardly into and through the needle when the slide moves the syringe plunger from the forward position to the retracted position. The wire advancing mechanism 134 is positioned to allow for the pre-threading or pre-positioning of other elements, like a dilator 102 and catheter 100 onto the wire before it is inserted into the workspace. The housing is configured to secure the catheter arrangement. This then presents the catheter for placement immediately upon placement of the wire. The wire advancing mechanism 134 also includes a loop guide 170 and is generally positioned between the slide 18 and housing 112 so as to provide forward translation of the wire proximal end with respect to the rearward or retracting movement of the slide 18 and syringe plunger. Specifically, referring to FIG. 5, the wire advancing mechanism 134 includes a loop guide 170 that has an input end or distal end 172 positioned to receive a wire from the wire gripping structure 30 of the slide 18 and an output or proximal end 174 for guiding a proximal end of the wire 24 and outputting the wire into a catheter 100 and through the catheter and ultimately into and through a needle mount 150 and then ultimately into and through the needle 132. The loop guide 170 may be a closed tube as illustrated in FIG. 5. Alternatively, the loop guide might be open with an appropriate configuration and structure for capturing wire 24 and guiding it through the loop guide from the distal end 172 to the proximal end 174. As illustrated in FIG. 5, the housing 12 may be appropriately formed to include mount portions 176, 178 that interface with and contain the respective distal end 172 and proximal end 174 of the loop guide 170. In the embodiment 110 of the device, because of the clearance needed for handling the catheter arrangement, the housing 112 includes an extension section 201 that extends rewarding on the housing to position the mount portion 178 rearwardly from the mount portion 176. In that way, space is created forward of the mount portion 178 to position the catheter arrangement 100, 102 forward of the wire advancing mechanism 134 so that the wire can pass through the catheter arrangement on its path to the needle 132. That is, the wire advancing mechanism sits rearwardly of the catheter arrangement. More specifically, the output or proximal end 174 of the mechanism 134 sits behind the catheter arrangement so a wire can exit the output end and enter the catheter arrangement directly. For securing the catheter arrangement, one or more additional mount sections 180 may be implemented in the housing 12 and or on the extension section 201 for holding a portion of the catheter arrangement. Referring to FIG. 5, the additional mount sections 180 may have slots 181 formed therein for receiving the elements that make up the catheter arrangement. The catheter arrangement may be snapped into the slots 181 or otherwise secured. In a similar fashion as device 10, negative pressure or a vacuum may be drawn through syringe body 14 by movement of the slide/plunger in the direction of arrow 62 and simultaneously, the proximal end 26 of the wire will advance in the direction of arrow 63 or in the direction of the movement of needle 32 into vasculature or another workspace as described herein.

In the embodiment of the device 110, the needle structure has to accommodate the fact that it cannot be slid off of and removed from the wire in the same fashion as with device 10. This is because the catheter arrangement 100, 102 sits behind the needle and prevents the needle from being slid rearwardly and removed from the wire. Rather, the device 110 implements a needle 132 and needle mount 150 that may be broken away or disassembled in order to be removed from the wire. More specifically, the needle mount 150 may incorporate a weakened section, such as along score line 151, that will allow separation of the needle mount into halves or smaller sections to remove it from the wire 24 to then allow the catheter arrangement to be slid down in the wire into position in the workspace. The needle 132 is also in the form of a peel apart or break apart needle for facilitation of the removal of the entire needle mount 150 and needle 132.

More specifically, device 110 includes a suitable needle mount 150 for coupling the needle 132 with the device. Particularly, the needle mount 150 couples the needle 132 simultaneously with both the syringe body 14 as well as the wire 24. Referring to FIG. 6E, the needle mount 150 includes a guide passage 152 for directing movement of the wire 24 into and through the needle 132. The needle mount 150 also includes a vacuum passage 154 that is coupled with the syringe body 14 to create a negative pressure in the needle when the syringe plunger is moved to the retracted position as illustrated in FIG. 6E. As described herein, movement of the slide and the syringe plunger to the retracted position draws negative pressure within the needle 132 through needle mount 150 to prevent the rearward movement of the slide and syringe plunger to the retracted position. Once the needle penetrates into the vasculature or other workspace through the tissue, the needle tip is no longer blocked or occluded and the negative pressure is broken so that air/fluid from a workspace is drawn into the needle and syringe body. The slide and syringe plunger may then be easily drawn rearwardly in the device 110 to the retracted position as fluid/air is pulled through the needle. The movement of the slide/plunger also simultaneously moves the wire gripping structure 30 rearwardly with respect to needle 132 and thus pushes wire 24 through the wire advancing mechanism 134. This turns or reverses or translates the wire movement and advances the proximal end 26 of the wire forwardly through needle 132 as illustrated in FIG. 6D.

In the embodiment of the invention illustrated in 5, the needle mount 150 creates an airtight passage in both the vacuum passage 154 as well as the guide passage 152. The needle 132 is dimensioned so the needle bore allows for passage of wire 24 therethrough in addition to the simultaneous passage of air and/or fluid rearwardly through the needle 132 when the wire 24 moves forwardly within the needle. The needle mount 150 forms an intersection point 160 for the vacuum passage 54, the internal passage 133 of the needle, and the guide passage 52 for the wire 24. The mount 150 therefore operates similarly to the needle mount 50 illustrated in FIG. 1. To that end, the needle mount 150 also includes a sealing mechanism 164 that engages with guide passage 152 and wire 24 for providing the airtight seal in the guide passage. That is, the sealing mechanism engages with wire 24 as it passes through the sealing mechanism and into the guide passage 152 so that wire 24 may move through the guide passage of the needle mount and through needle 32 at the same time that negative pressure and/or fluid and air is being drawn through movement of the plunger 20 within the syringe body 14 as shown in FIG. 6E.

In accordance with one aspect of the invention, the housing 112 and syringe body 14 may be formed as a unitary structure wherein the housing is part of the syringe body. In another embodiment of the invention, the housing is configured to couple with a separate syringe body with the syringe body in a proper position with respect to the housing grip portions 16. Similarly, the slide 18 may be formed together with a syringe plunger as a single piece such that the slide and wire gripping structure are moved as the syringe plunger is moved. Alternatively, the slide 18 may be formed to couple to a separate syringe plunger in order to couple the movement of one to the other and the movement of the wire with the plunger. In that way, the device with a housing 12 and slide 18 might utilize a typical syringe having a syringe body and syringe plunger as shown in FIG. 1A.

FIGS. 6A-6J illustrates implementation use of the device 110 as illustrated in FIG. 5. Device 110 provides an all-in-one solution for delivery of the guide wire, catheter, and other elements to a target space. Referring to FIG. 6A, in use, the device 110 is loaded with a catheter 100, a dilator 102, and other elements, such as ports 104 that are preloaded or positioned on wire 24. As noted, the example and elements shown in FIG. 6A is not limiting and other elements might be preloaded onto wire 24. The housing 112 includes the mount sections 180 into which the catheter assembly may be loaded. For example, the mount sections 180 might clip on to catheter 100. The wire 24 can then be threaded through advancing mechanism 134 and through the catheter arrangement and into needle 132. The wire gripping structure 30 can be manipulated to grip the distal end, while the proximal end of the wire is threaded into the needle 132. FIG. 6B shows the device 110 loaded with a catheter arrangement and wire such that the catheter arrangement and the wire is in line with the housing 112 for performance of the procedure utilizing device 110. As illustrated in FIG. 6C, as the device 110 is used the grip portions 16 and 22 are engaged and pressure is applied to the slide 18 and grip portions 22 by movement of a user's finger in the direction of arrow 62. The wire 26 may be advanced into the target space. As discussed herein with respect to FIG. 4B, the wire will advance into the target space once the negative pressure is released and the plunger and the slide 18 are able to slide more freely rearwardly with respect to the syringe body and the housing 112. As shown in FIG. 6D, the wire will advance in the direction of arrow 63 as the slide 18 is moved further in the opposite direction toward a retracted position. This then positions the guide wire, in line with the catheter arrangement, for further insertion of the catheter arrangement into the target space. Referring to FIGS. 6E and 6F, the device 110 may be removed from the catheter arrangement and wire 26 once the wire is positioned into the workspace.

FIGS. 6E and 6F illustrate that once the wire has been positioned appropriately within the target space after penetration into the target space by the needle and the drawing of any air or fluid into the syringe body 14 as illustrated by arrow 15, then the device 110 is no longer needed. The catheter arrangement may be removed from the device 110. The device may be removed from the needle 132 and needle mount 150. FIG. 6F illustrates an exposed catheter 100, a dilator 102, and wire 24 with device 110 removed. The needle mount 150 remains in position with needle 132. Because of the in-line position of the catheter and dilator on and behind the wire 24 portion of the workspace, the needle mount 150 and needle 132 cannot simply be slid off the end of wire 24. To that end, in the use of device 110 with both the wire and in-line catheter arrangement, needle mount 150, and needle 132 must break away or tear away from wire 24.

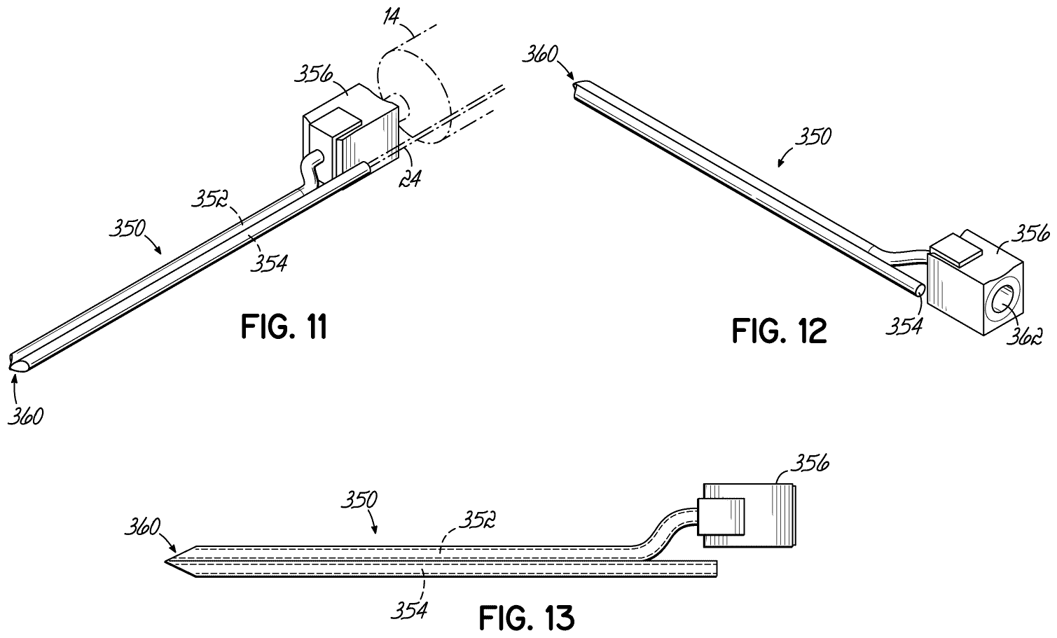

Referring to FIG. 6G, once the needle and wire have entered workspace 84 through tissue 82, such as through an entry point 83, the needle may be withdrawn as shown by arrow 85 and only the wire 24 left in position within the workspace 84. However, the needle mount 150 and needle 132 must be broken apart to allow the advancement of catheter 100 and dilator 102 or any other element of the catheter arrangement on wire 24. The catheter arrangement can then progress in the direction of arrow 87 as illustrated in FIG. 6H. To do so, the ends of the needle mount 150 may be grasped and torn apart as illustrated in FIG. 6H. Generally, the needle 132 will be a breakaway or peelable needle that may be split or have at least one side pull off to free wire 24. The needle mount will also be weakened, such as along the score line 151, to be separated as shown in FIG. 6H. The two parts of the needle mount 150a and 150b, along with the respective halves of the needle 132 may be pulled apart in the direction of arrows 89 as shown in FIG. 6H. If a double barrel needle is used, as shown in FIGS. 11-13, one side of the needle might be peeled off. Then the needle and mount are removed. The dilator 102 and catheter 100 may be slid along wire 24 into workspace 84, such as through the same tissue entry point 83 that the needle created. Referring to FIG. 6I, the dilator 102 and catheter 100 are shown in position along wire 24 within the workspace 84. Once the catheter 100 is properly positioned, the wire 26 and dilator 102 may be withdrawn as illustrated in FIG. 6J and then the catheter 100 and any respective ports 104 are in position and may be utilized for the desirable medical procedure.

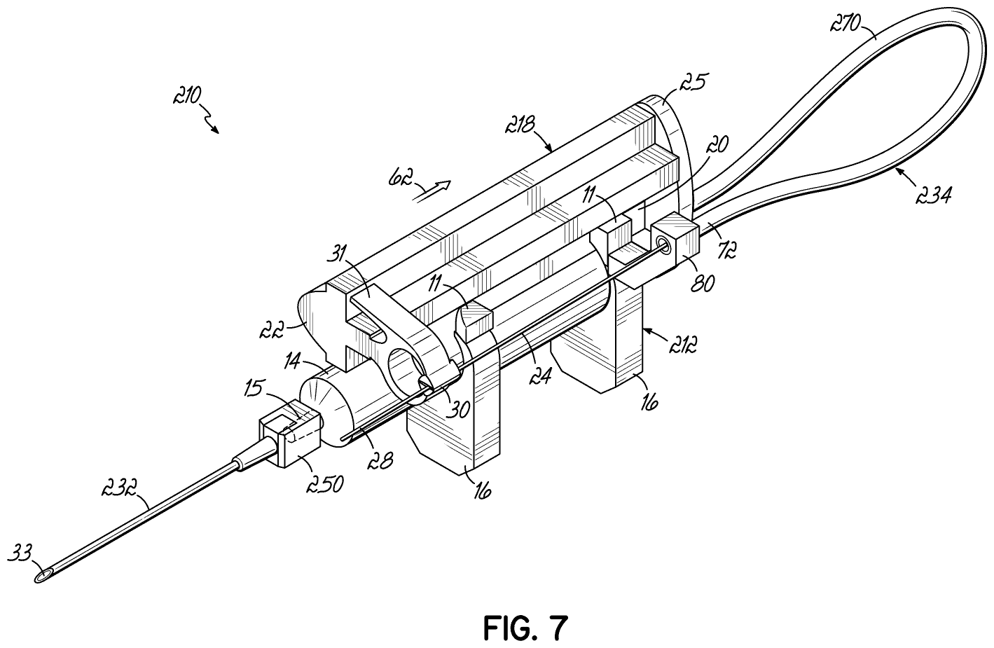

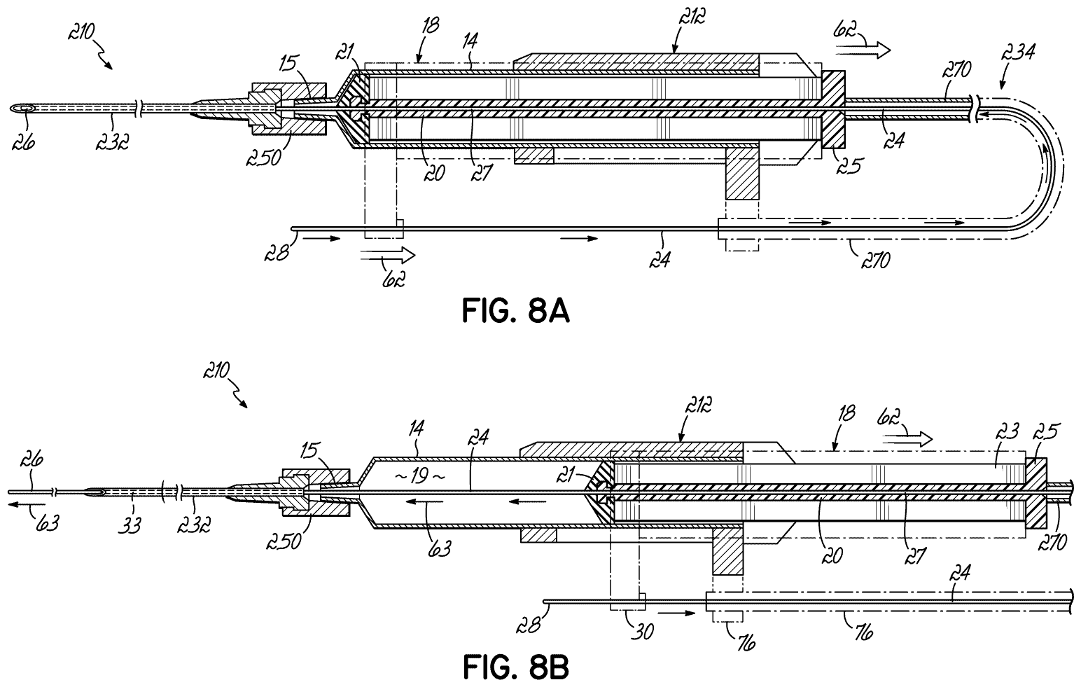

One advantage of the preloaded catheter arrangement in FIG. 5 is that a stop structure 13 might be placed on a back end or distal end of the wire. Such a stop structure 13 may prevent the catheter arrangement from falling off a back of the wire or may prevent the back end of the wire from getting pulled past the skin. Since the needle and mount break or peel away, thus removing the need to slide the needle off of the wire, the stop structure may be implemented. Other embodiments of the invention may utilize wire advancing mechanisms that are positioned and operate differently, as well as alternative syringe bodies and needles and needle mounts for advancing the wire 26 in the opposite direction from the movement of the plunger. Specifically, referring to FIGS. 7-8B, an alternative device 210 is implemented that has a housing 212, grip portions 16, a slide 218, and wire gripping structure 30 for the movement of a syringe plunger and wire simultaneously. However, in the embodiment of the device 210 illustrated in FIGS. 7-8B, the wire advancing mechanism 234 is configured to present the wire distal end 24 to proceed through the end 25 of the slide 18 and through plunger 20. As illustrated in FIG. 8A, the plunger 20 may include an internal passage 27 through which wire 24 may progress. As illustrated, the loop guide 270 of the wire advancing mechanism 234 may terminate at the end 25 of the slide which coincides with the end of the plunger and the wire may be guided through the syringe plunger 20 and the syringe body 14 to engage with the needle mount 250 and the needle 232 as the slide 18 and plunger are moved in the direction of arrow 62. As illustrated in FIG. 8A, the wire 24 and particularly the distal end 28 of the wire moves with slide 18 and its direction is changed by the wire advancing mechanism 234 to proceed through the syringe plunger 20, syringe body 14 and through the needle 232 into the appropriate workspace as illustrated in FIG. 8B. That is, as the slide and plunger move in the direction of arrow 62, the wire moves in the direction of arrow 63 through the needle 232 as discussed herein with various of the embodiments.

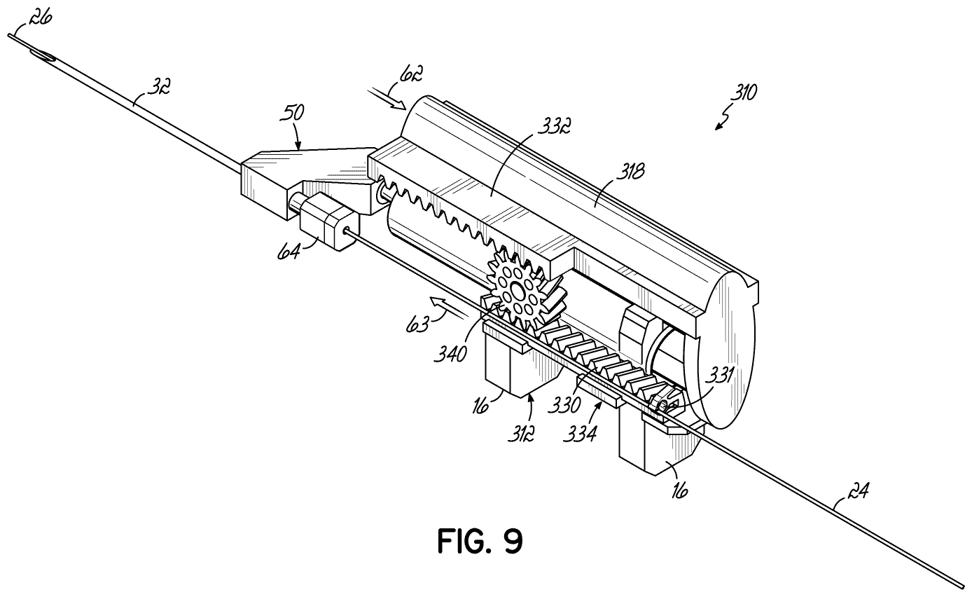

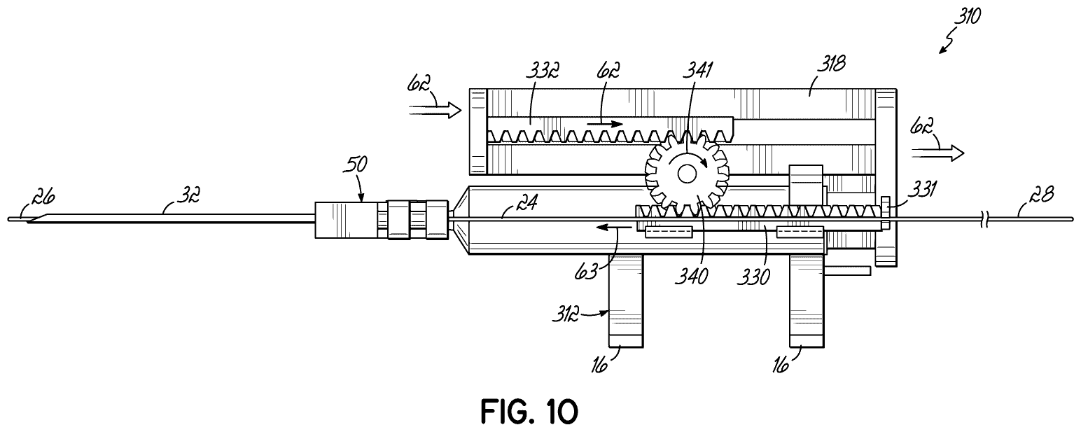

In accordance with an alternative embodiment of the invention, different wire advancing mechanisms may be utilized. FIGS. 1 and 5 illustrate one version having a loop guide. FIGS. 9 and 10 illustrate an alternative version which is configured for actively advancing the proximal end of the wire forwardly into a needle of the device when the slide moves the syringe plunger from the forward position to a retracted position. In the embodiment of the device 310 as illustrated in FIGS. 9 and 10, the advancing mechanism is an active mechanism, rather than the passive mechanism as with loop guide 70. Specifically, the housing 312 is configured to include a movable rack gear 330. The slide 318 is configured to include an opposing rack gear 332. The rack gear 330 is configured to translate along housing 312 in the direction of arrow 63 when the slide 318 and rack gear 332 are moved to a retracted position in the direction of arrows 62 as shown in FIG. 10. A pinion gear 340 is rotatably mounted with respect to the housing 312 between the two rack gears 330, 332. The pinion gear rotates in the direction of arrow 341 as the rack gear 332 on the slide 318 is translated during movement of the slide and plunger. In turn, the pinion gear 340 acts on the movable rack gear 330 that is translated in the direction of arrow 63. The movable rack gear 330 includes a wire gripping structure 331 which grasps the distal end 28 of the wire 24. As the slide 318 and plunger are moved to a retracted position, the movable rack gear 330 and wire gripping structure 331 are translated in the direction of arrow 63 and thereby translates the wire 24 through needle mount 50 and needle 32 in accordance with features of the invention. Therefore, the wire advancing mechanism 334 as illustrated in FIGS. 9 and 10 shows an embodiment that incorporates active forward movement of the wire upon retracting the plunger, rather than a passive change of direction. As such, the wire advancing mechanism as illustrated and described herein may actively move the wire, rather than passively reverse its direction from the direction of the slide and plunger. One additional use of the active or geared version of a wire advancing mechanism is that the rack gears and pinion gears might be adjusted and configured to increase the feedback to a user such that hand movement in moving the slide and plunger to the retracted position might be adjusted in both the speed of the wire movement (increased or decreased) in the workspace and the force feedback of the wire movement (increased or decreased) to the user's hand. The current use of the passive wire advancing mechanism might provide a 1:1 ratio of hand force and wire movement force. The active wire advancing mechanism as shown in FIGS. 9-10 might provide a higher or lower ratio of forces based on gearing, for example.

FIGS. 11-13 illustrate an alternative embodiment of a needle mount and needle in accordance with an aspect of the invention. The needle mounts 50 and 150 as illustrated in FIGS. 1 and 5, for example, incorporate a sealing mechanism 64 for providing a seal with respect to the movable wire 24 so that negative pressure may be drawn through the needle mount of the syringe body 14 and plunger 20 through the bore of the needle as the wire moves through the same bore. Alternatively, a needle with multiple passages may be implemented such that the passage used to draw the negative pressure is different than the passage which advances the wire. Referring to FIGS. 11-13, a double needle is illustrated for that purpose. Specifically, the double needle 350 incorporates a needle portion or passage 352 for drawing the negative pressure or vacuum utilizing syringe body 14. A separate needle portion or passage 354 receives wire 24 for advancement upon moving a slide/plunger to the retracted position in accordance with the invention. To that end, the needle mount 356 is configured such that negative pressure may be drawn through needle passage 352 while the wire 24 simultaneously advances through needle passage 354. To that end, the passages 352, 354 are generally parallel and share a common pointed tip 360 for penetration into tissue and a workspace. Needle mount 356 incorporates an appropriate aperture 362 for coupling with the tip of the syringe body 14 for initially drawing a negative pressure and then subsequently releasing the slide/plunger upon the proper placement of the needle to move the plunger to a retracted position and move the wire 24 forwardly into the needle.

FIG. 14 illustrates another alternative embodiment of a needle mount and needle arrangement for a device of the invention. The needle mount 370 mounts the needle 32 to be inline with the syringe rather than inline with the advancing wire. To that end, the vacuum passage 374 is straight and feeds into the tip 15 of the syringe. The wire guide passage 372 is arced or otherwise angled to simultaneously feed the advancing wire into needle 32 in accordance with the invention.

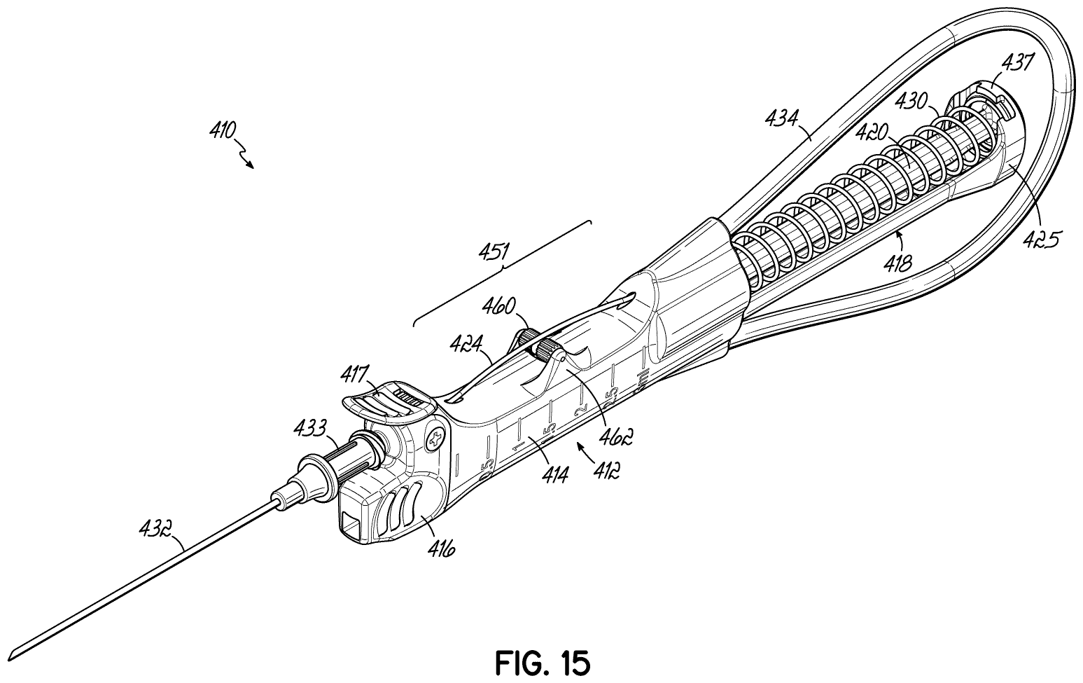

FIG. 15 illustrates an alternative embodiment of a device 410 for advancing a wire into a body tissue space or workspace, such as the vasculature of an animal like a human, to provide vascular access. While the examples are discussed with respect to placement of a wire in vascular cavities, the invention may be applied to other body tissue cavities and workspaces. Such wire advancement can be used for a number of different medical procedures as noted. Therefore, as noted, the term vasculature is used to generically refer to a space or workspace in tissue in a body, and the use of the invention is not limited to just wire placement in vasculature. Furthermore, while the embodiments are disclosed for placement of a guide wire to position other elements in the body workspace, other wire structures may be inserted with the device to provide body cavity access, and so the element is referred to herein generally as a “wire”.

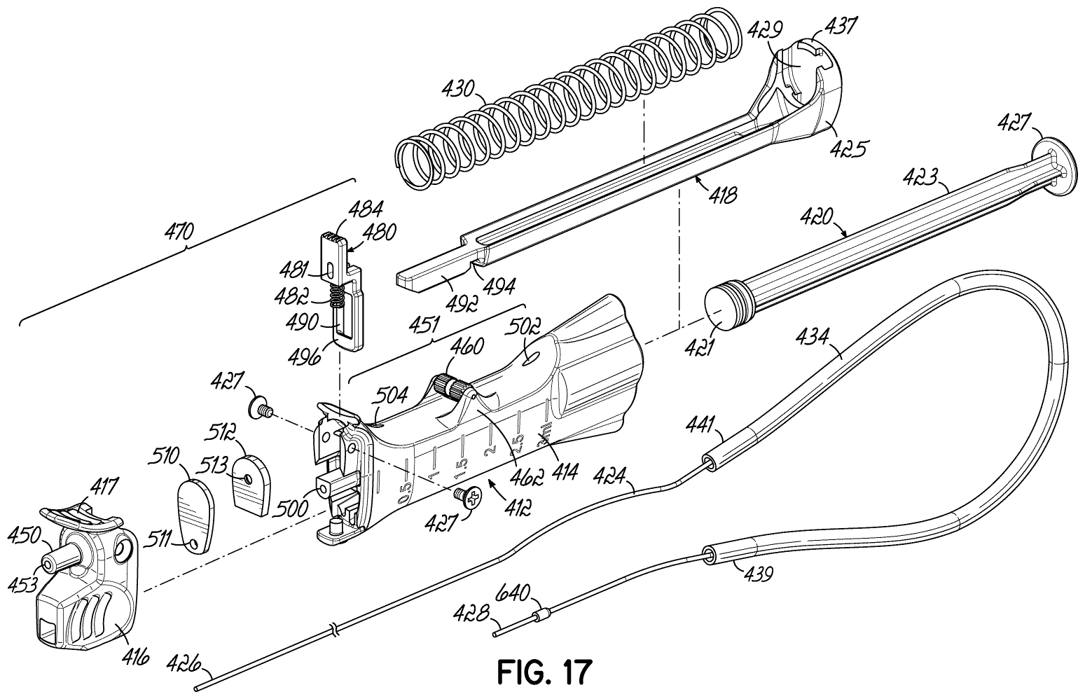

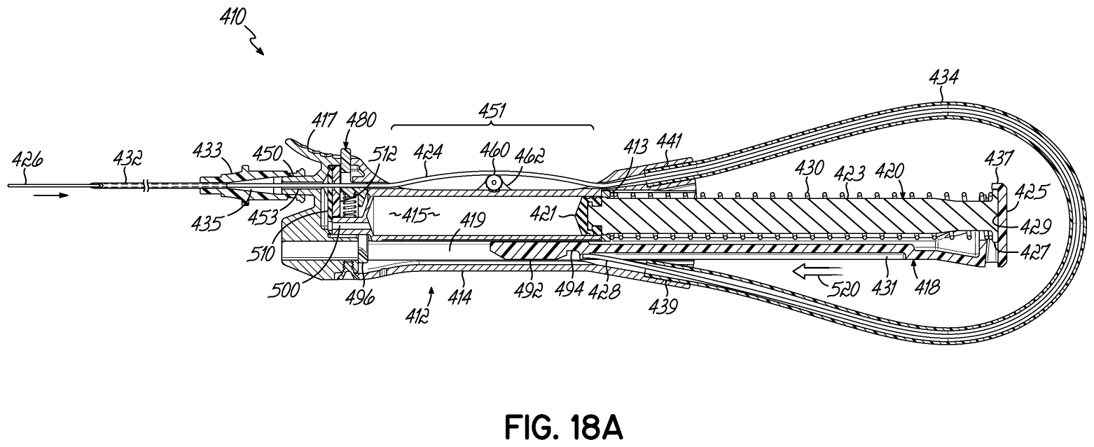

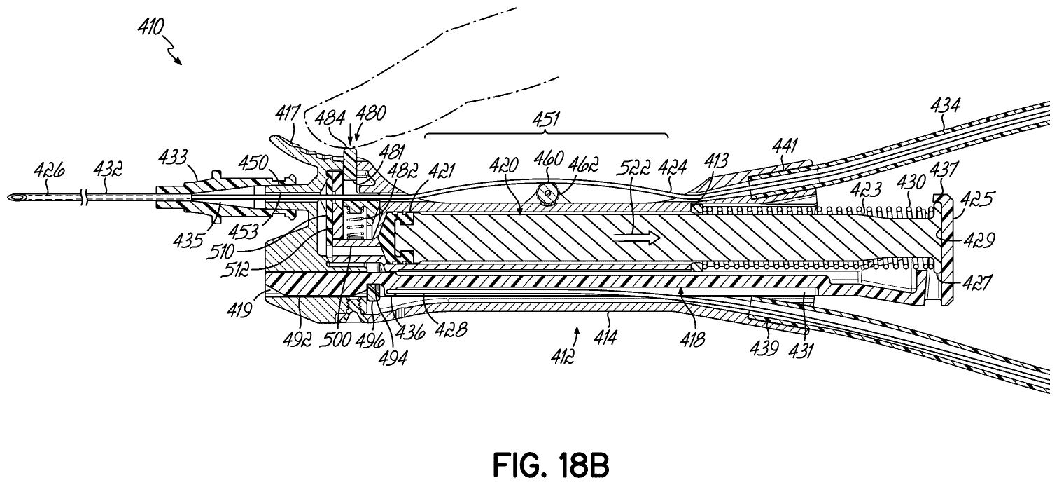

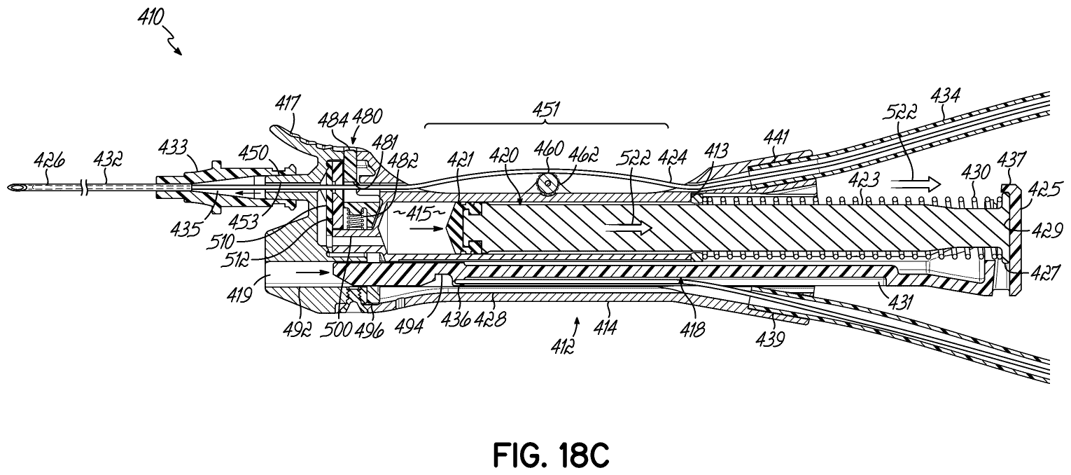

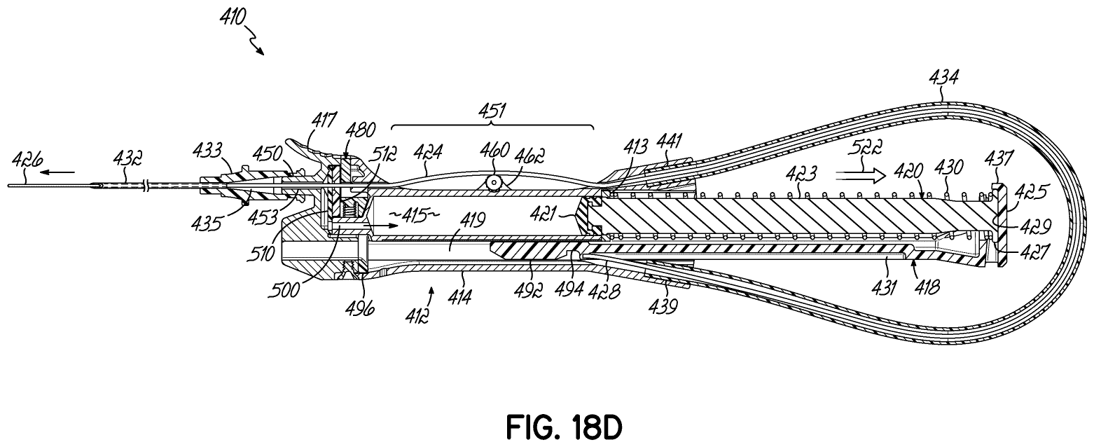

Referring to FIG. 15, device 410 includes a housing 412 to be held and manipulated. In accordance with one feature of the invention the device housing is designed to be gripped and operated with a single hand. It may be held and operated by either the right or left hand of the user and may be held upright as shown in FIG. 15 or may be turned upside down. The housing is configured for effectively providing a syringe body 414 in accordance with a feature of the invention that uses a syringe and vacuum created by the syringe plunger to provide activation and advancement of a wire into a cavity. While other embodiments of the invention, as disclosed herein, interface with and operate with separate syringe and plunger elements, various of the syringe elements of the device 410 are provided by or built into the housing 412. In the illustrated embodiment, for example, the housing is configured to effectively provide the syringe body portion or syringe body 414 (see FIGS. 17 and 18A). That is, the housing portion 414 acts as a syringe and creates essentially a passage or cavity 415 to receive a syringe plunger 420 so that the plunger can move in the housing 412 similar to the movement of a plunger in a typical syringe or syringe body. A syringe body having a capacity of 3 ml might be used.

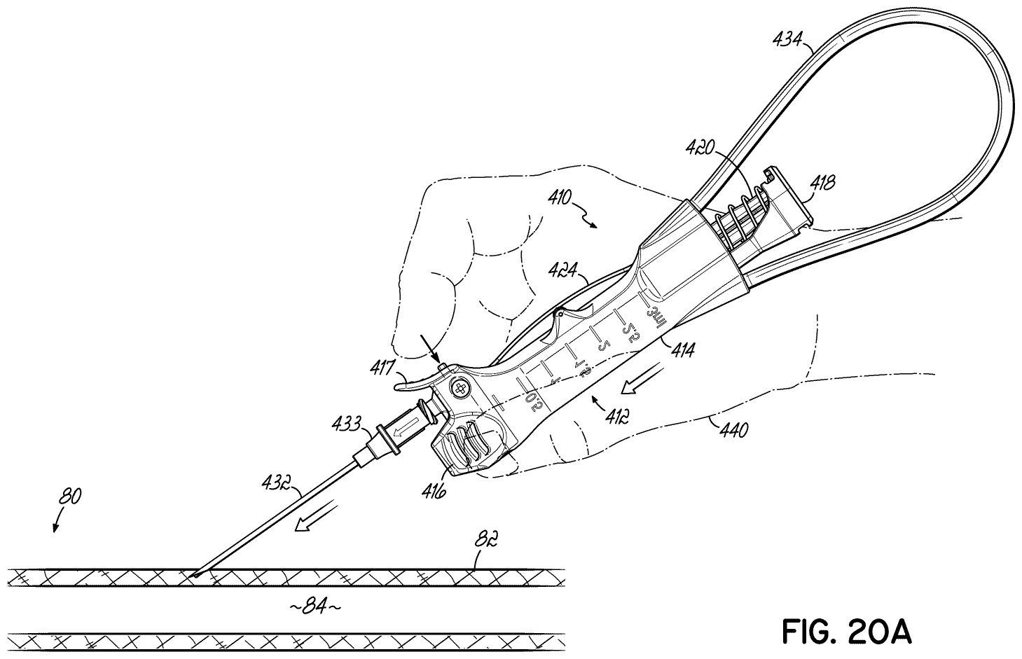

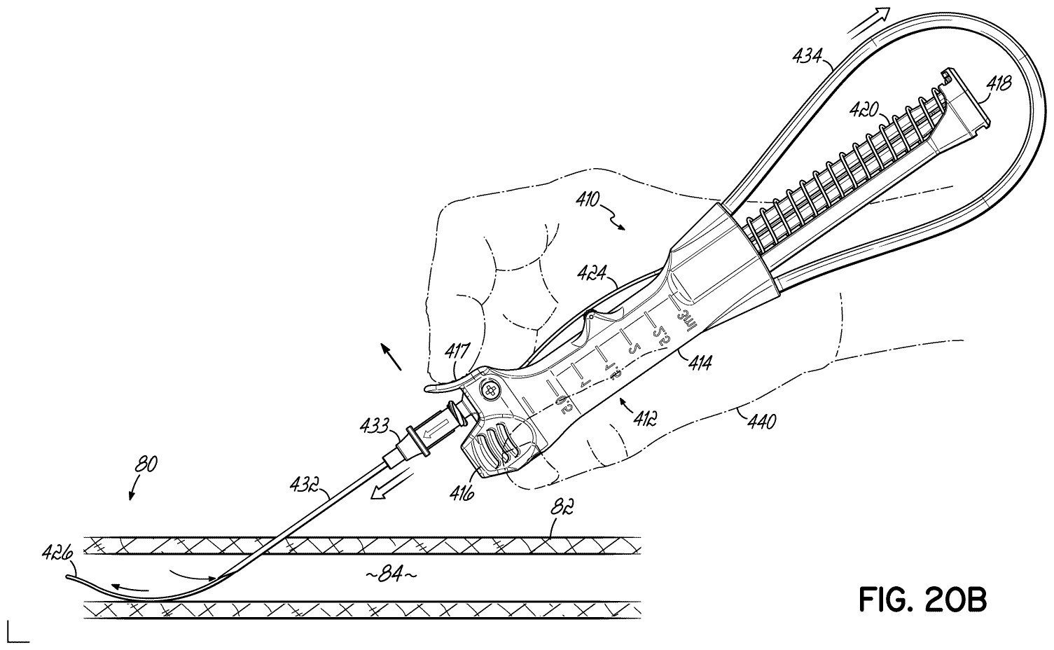

The housing 412 is also configured for engagement by a user for gripping the device. For example, the device 410 and the housing 412 include one or more grip area 416s and an activation area 417 that may be engaged, for example, by one or more fingers of a user to position and activate the device in use (See FIGS. 20A, 20B). The housing provides a way of gripping the syringe body for positioning the device and then activating the device in use as discussed herein. For example, as shown in FIG. 20A, the grip areas 416 may be gripped by the thumb and finger and activated with another finger at area 417. Alternatively, the housing might be held by the fingers and activated in the area 417 by the thumb. The device 410 may be held in various positions and various different orientations as noted. It also can be handled in the either the right of left hand and manipulated and activated but a single hand. In that way, the other free hand may be used to manipulate other devices or elements, such as an ultrasound probe, for example.

A slide element or slide 418 is positioned to move in the housing 412 and specifically is positioned in an appropriately formed channel 419 in the housing to move between a forward or primed position and a retracted or activated position (see FIGS. 18A-18D). The slide element 418 is configured for coupling with the syringe plunger 420, that is also configured to move in the housing 412, and specifically move in the syringe body 414 formed in the housing 412. (see FIG. 18A) In accordance with the disclosed embodiment, the plunger 420 and slide element 418 are configured to move together. The syringe plunger 420 may be configured to resemble a typical syringe plunger having an elongated body portion 423 which is terminated in a rubber or plastic stopper 421 that seals with the internal wall of the syringe body 414 and creates an air and fluid tight seal.

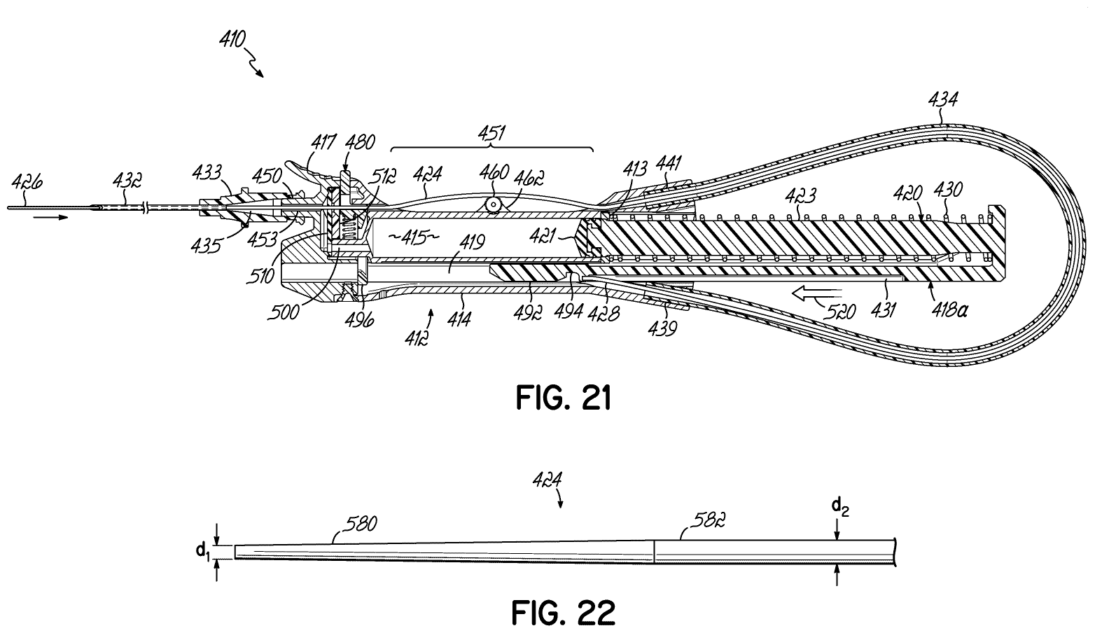

As noted, the slide 418 and plunger 420 move together. To that end, the slide 418 may be formed and configured for coupling with an end 425 of the syringe plunger. That is, the slide 418 may be formed and configured to couple to a separate syringe plunger 420 in order to couple the movement of one to the other. Alternatively, the slide 418 may be formed together with or as an extension of a syringe plunger 420 as a single or unitary element, such that the slide moves as one piece with the syringe plunger as shown in FIG. 21. FIGS. 15-17 show one embodiment of the device 410 wherein housing 412 and syringe body 414 form the generally cylindrical passage 415 configured for receiving syringe plunger 420. The slide 418 is configured to slide in the housing 412 and is positioned generally parallel to the syringe body 414, passage 415 and plunger 420 as shown in FIGS. 18A-18D.

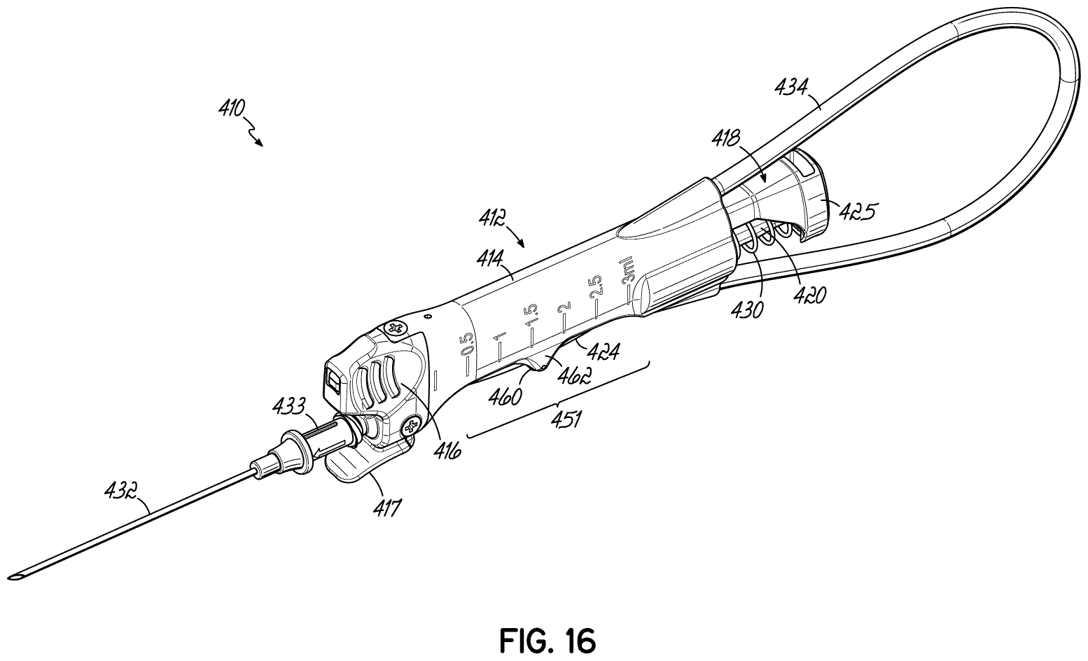

In the embodiment in FIG. 17, the slide 418 has an end 425 that is configured to couple with end 427 of the plunger so that the plunger 420 lies along the slide as shown in FIG. 15-16 when they move together. The slide end in the disclosed embodiment has an indent 429 or other structure for engaging end 427 of syringe plunger 420 to hold the end 427 and couple the slide and plunger for movement. The plunger and slide engagement at the ends 425, 427 may be a friction fit or the plunger end 427 might be held in the indent 429 by one or more tabs 437 that snap on the end 427. In that way, the slide acts as an extension of the plunger to move or draw the plunger rearwardly in housing 412 as discussed herein.

As disclosed, the housing 412, syringe body 414, slide 418, and syringe plunger 420 may be formed of a suitable material, such as a medical grade plastic material that is lightweight and may be sterilized. The material provides sufficient rigidity for movement of the plunger in the housing 412 as well as holding and guiding of a wire 424.

For movement of the slide 418 and plunger 420, and the end of wire 424, the embodiment of FIGS. 15-16 includes a bias force element that provides a biasing force to move the slide and plunger from a primed or cocked or forward position to an activated or released or retracted position as discussed herein. In the illustrated embodiment, the bias force element is a coil spring 430. In FIG. 15, the coil spring 430 is positioned between an end 413 of the housing 412 and the end 425 of the slide 418. The slide 418 may be pressed along with the plunger 420 from the released or activated position as shown in FIG. 15 and toward the housing 412 to the primed or cocked position as shown in FIG. 16 with the spring 430 being compressed to prime the device 410 as shown in FIG. 16. In the embodiment of FIGS. 15-16, the compression of the spring 430 will create a biasing force in the opposite direction of the housing to push on or bias the slide and plunger back to an activated or released position as shown in FIG. 15 as discussed herein. Another spring element, other than a coil spring, might be used and compressed as illustrated to prime and activate the device.

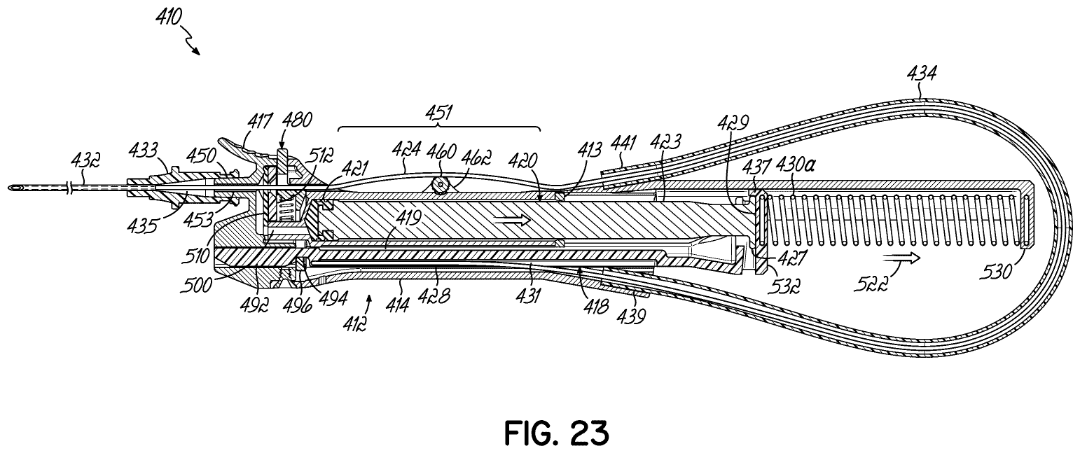

In an alternative embodiment, as shown in FIG. 23, the bias force element and housing are configured for creating a pulling force, rather than a pushing force, on the slide and plunger. Referring to FIG. 23, the housing 412 includes an extension portion 411 that extends rearwardly of the syringe body 414 as shown. The extension portion 411 includes an anchor end 530 that couples with one end of the bias force element. The other end of the bias force element couples with the end 532 of the slide and the coupled plunger. In the embodiment of FIG. 23, the bias force element is a coil spring in the form of an extension spring 430a that can be stretched or extended to prime or cock the device rather than being compressed as shown in other embodiments. The bias force is to pull the slide and plunger rather than push them as with a compression spring. Then, when the device is activated, the spring 430a will retract to pull (rather than push) the slide and plunger. In FIG. 23, the device is shown in the primed position with the spring 430a extended and the slide in a locked position ready to be activated. Once the device is activated and the slide is free to travel, the extension spring retracts to a shorter, coiled position to pull the slide and plunger and create a vacuum force and eventually forward movement of the wire. The illustrated embodiments show coil springs, but other spring element, like a solid spring, a flexible band or other resilient bias force element might be used to present a force to push or pull and move the slide and plunger from the primed position to the activated position.

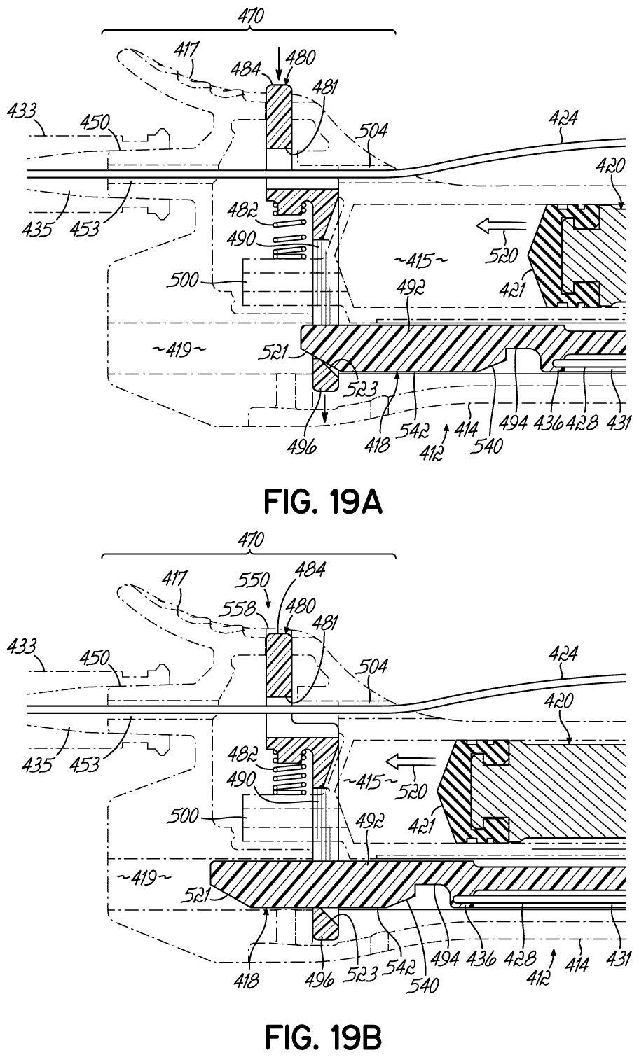

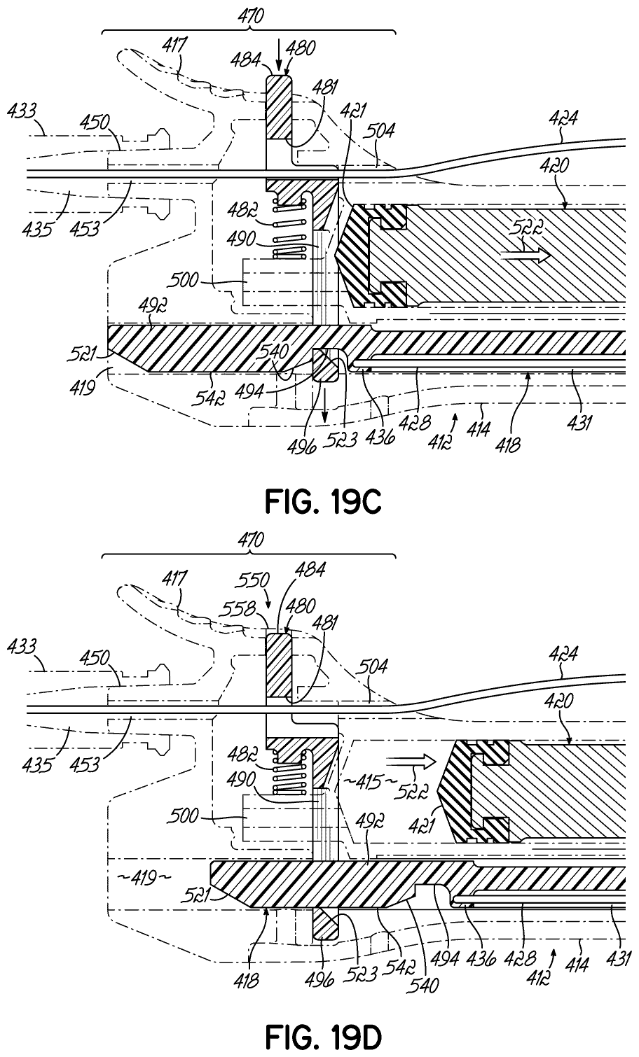

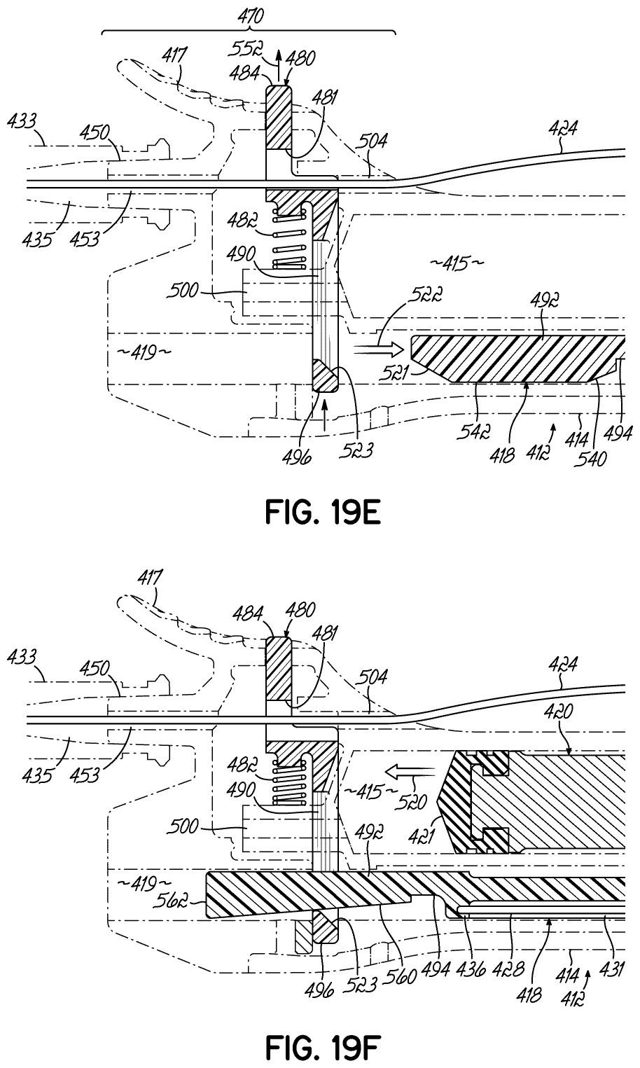

Device 410 manipulates and includes a wire 424 for delivery and placement into a tissue workspace. The wire 424 includes a proximal end 426 and a distal end 428. The proximal end moves through an outlet 450 of the housing that interfaces with a needle 432. The slide 418 is releasably coupled with the distal end 428 of the wire for moving the distal end and thereby advancing the proximal end 426 forwardly in the needle 432 when the slide and plunger move from a forward or primed position of FIG. 16, to an activated position of FIG. 15. In one embodiment, as illustrated in FIGS. 18A and 19A, the slide 418 includes a channel 431 and a pocket 436 formed in the slide that releasably engages or contains the distal end 428 of the wire for facilitating movement of the wire with the slide and plunger as described herein. The containment in the slide and pocket in the housing prevents the wire from being dislodged or pinned by fingers to prevent advancement. A needle 432 is coupled with the outlet 450 of syringe body 414 and is also configured to have a bore for guiding the proximal end 426 of the wire into vasculature or another workspace in accordance with features of the invention.

More specifically, movement of the slide 418 and coupled syringe plunger 420 rearwardly in the housing to an activated position and away from the needle 432 in the syringe body 414 will draw negative pressure in the needle. At the same time, the spring bias of the slide 418 that releasably grips the wire in the channel pocket 436 will also seek to advance the wire 424 through needle 432 into the vasculature (see FIGS. 18C, 18D) once the pressure is released upon the needle entering a vascular space or cavity. The channel pocket 436 of the slide 418 holds or contains the end of the wire and pushes it in the direction of the biased slide away from the needle.

To then translate such movement of the distal end 428 to a forward movement of the proximal end 426 through the needle, a redirection mechanism or wire advancing mechanism 434 is coupled generally between the slide 418 and housing 412 as seen in FIGS. 15-16. The wire advancing system acts as described herein with respect to the embodiment in FIG. 1. Namely, the wire advancing mechanism 434 acts on the wire to translate movement in one direction into movement in another direction. The purpose of the wire advancing mechanism 434 is to reverse or translate the rearward or retracted movement of the slide 418 and wire end 428 with the accompanying movement of the syringe plunger 420 from the forward or primed position of FIG. 16 and create a forward movement of the proximal end 426 of the wire 424 in one movement stroke. This advances the proximal end 426 of the wire forwardly into and through needle 432 when the slide 418 and the syringe plunger 420 move to a rearward or fully released position as illustrated in FIG. 15. A distal end 439 of the wire advancing mechanism interfaces with the housing to receive the wire from the slid 418 as shown in FIG. 18A. The wire is then directed toward a proximal end 441 of the wire advancing mechanism that interfaces with another portion of the housing to deliver the wire out of the housing and to exposed area 451 and then to the needle. Although the wire advancing mechanism 434 is shown, a gearing system, such as is shown in FIG. 10 might be used to interface with the moving slide or other moving portion to translate the wire forwardly with a rearward movement of the slide or plunger.

The wire 424 may include one or more markings so that a user may track the forward movement of the wire. The wire advancing mechanism 434, for example, might be transparent to see the markings as the wire moves forward in the tissue space or cavity.

As illustrated in FIGS. 20A and 20B, a hand 440 of a human operator engages the respective grip area 416 and activation area 417 of the housing 412 and slide 418. As discussed further herein, the device is primed with the biasing force element, such as bias spring 430, in a primed or compressed position as shown in FIG. 20A. The primed device is positioned, and particularly the needle 432 is inserted into tissue, such as vascular tissue. The device may then be activated by the finger of the user as shown, thus releasing the slide and plunger to be acted upon by the biasing force of spring 430 (See FIG. 18B). The negative pressure or vacuum created at the needle end when blocked by tissue will prevent the syringe plunger 420, and slide 418, from full activation as shown in FIG. 18C and the device will stay partially primed and biased to continue to draw a vacuum. As long as the needle is blocked by tissue, that negative pressure will generally prevent significant expansion of the spring element and rearward movement of the slide and plunger. The slide and plunger may move slightly as shown in FIG. 18C to create the negative pressure but will be prevented from moving readily to the full activated position. Once the needle tip penetrates through the tissue and the vasculature and the negative pressure is broken with a flow of blood or fluid through the needle and into the syringe body, the slide and syringe plunger will move to the activated position as shown in FIGS. 18D and 20B. This, in turn, moves the wire 424 and particularly the proximal end 426 into a vascular space as shown in FIG. 20B.

In that way, the device 410 of the present invention provides a single motion and unique biasing force and smooth forward movement of the wire through needle 432 and into the vasculature as the plunger is moved to the activated position and a negative pressure is drawn and released with respect to the syringe plunger 420 moved by slide 418. With a biasing force, the user is free to focus on manipulation of the device and needle and can selectively activate the device from the primed position once the needle is positioned in tissue. Once activated, the user can guide and further insert the needle until it enters a vascular space. Once the end of the needle 432 enters a vascular space or cavity, the flow of blood into the needle allows the syringe plunger 420 and slide 418 to extend to full activation under the force of spring 430 (see FIG. 18D). The biasing force can then take over to move the slide 418 and plunger 420 and push wire 424 in out of the end of the needle 432 in a single stroke or movement as described further herein and shown in FIG. 18D.

The needle 432 is configured for coupling with the syringe body 414 and is coupled with a base or lumen 433 that is configured to be press fit or screwed onto outlet 450 of the housing. The base 433 has an appropriate bore 435 therethrough for passage of wire 424 into the needle and passage of fluid into the housing and the syringe body. A luer lock arrangement might also be used for coupling the needle and housing.

Once the wire is in the vascular space, and the slide/plunger are fully activated with the full extension of spring element 430, the wire 424 may be further moved or manipulated by the user. To that end, in accordance with one feature of the invention, a portion of the wire is exposed, as shown in area 451. Through that exposure, a user may further manually push or manipulate the wire in the vascular space. Furthermore, the exposure of the wire to the device user will allow the user manipulate the wire while advancing it, such as by twisting or torquing it as need to ensure that the tip and wire advances properly in the tissue space and does not get hung up. For example, sometimes the wire will run into stenoses or turns, and manipulating the tip by twisting the external portion of the wire can allow the wire to progress around them. The disclosed embodiment of device 410 has a roller feature over which the wire section in area 451 may roll for ease of further pushing or manipulation. Specifically, as shown in FIGS. 15-17, a roller element 460 is rotationally mounted on a mount 462 that presents the roller element 460 above a surface of the housing 414 to roll with the movement of the wire. Specifically, as shown in FIGS. 15 and 18A-18D, the roller 460 includes a channel 464 that captures the wire 424 therein as the wire passes over the roller element. A user, when holding the device, may move a finger or thumb over the wire and roller element and roll the roller element forward to further push the wire into the vascular space or rearward to withdraw the wire. Or, as noted, the user may move their thumb or finger side to side on the roller for twisting the wire. Once the wire is in the proper position in the vascular space, the device 410 may be pulled from the wire, with the roller being used to advance the wire. The user can advance the wire until a desired length of wire is within the vasculature. Once the wire is manipulated and in the desired position, the device may be pulled therefrom so the wire is exposed for being used to guide other components and devices into the vascular space in accordance with various medical procedures. In accordance with one embodiment of the invention, the device is withdrawn after use and the wire passes passed out of the device 410. In another embodiment, the wire might be captured by the device and pulled out of the vascular space when the device is pulled away, as discussed below in use with a catheter.

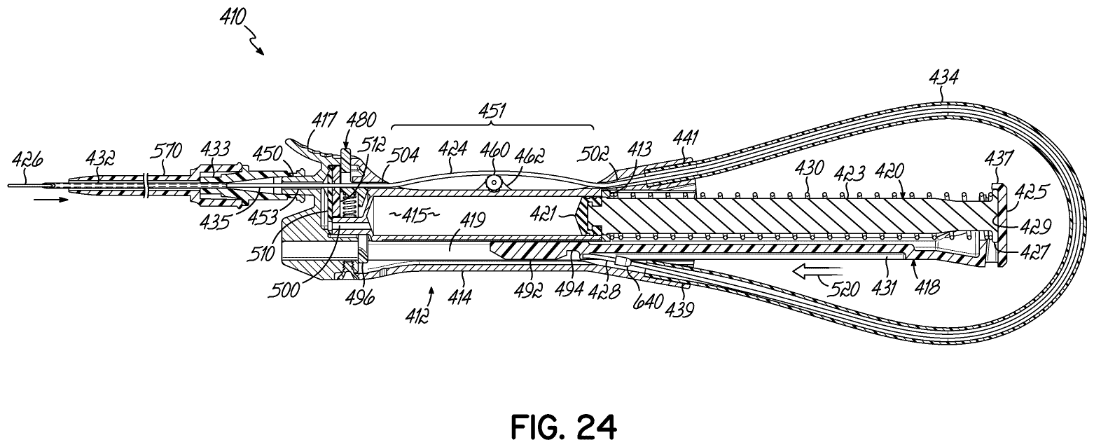

In one embodiment of the invention, as shown in FIG. 24, a catheter device 570, such as a radial or arterial or peripheral intravenous catheter, might be preloaded onto the needle. Once the needle is in the vessel with the wire advanced, the user may twist the device 410 and needle 180 degrees or in some fashion so that the sharp needle tip is now on the top of the wire and will not get stuck on the tissue the user can then advance the entire needle and catheter device 570 over the wire until the catheter is all the way into the skin. The needle and wire can then be withdrawn. This can be done with one hand while holding a device, such as an ultrasound probe in the other hand. In accordance with still a further embodiment of the invention, when a catheter is used off the needle as shown in FIG. 24, a wire stop structure 640 may be implemented. Such a stop structure 640 is shown, for example, in FIGS. 17 and 24. The stop structure 640 is sized and configured for being captured in the housing or with the housing so as to stay with the device 410 when the needle and device are withdrawn when using the catheter option shown in FIG. 24. That is, when catheter 570 has been slid over wire into position, the device and needle may be pulled from the site. The stop structure 640 may proceed through wire advancing mechanism 434, but may be sized to catch in the housing, such as in bore 502 or in bore 504 so as to be drawn out of the tissue when the needle and device housing are withdrawn. If for some reason it would be desirable to move further elements along the wire once a catheter is in position, the stop structure might be popped or slid off the wire to facilitate the wires further use. When a catheter is not in use, a stop structure would not normally be needed, so the FIG. 17 only shows the structure for illustrative purposes as in many uses of the present invention, a stop structure would not be placed on the wire.

In accordance with one feature of the invention, the device 410 provides a tactile feedback in its operation that assists the user with knowing the status of the procedure by feel in one hand. To that end, the button element, such as a button slide 480 interacts with the slide and particularly the latch end 492 of the slide that has a unique shape for a unique interaction with the button slide 480. When the device is primed and ready for use, the button slide 480 and particularly the button portion 484 sits raised above the activation area 417. To activate the device, the button portion 484 is depressed and the button slide travels over the latch end to be drawn down below the activation area 417. The needle is positioned, and when a tissue space is found and the device can fully activate, the button slide will move off of the latch end and is biased to snap back upwardly to the activation area 417 and strike the finger or thumb of the user to let them know that the slide has pulled back to draw in blood and wire is advanced.

FIGS. 19A-19E illustrate an activation mechanism 470 in accordance with one embodiment of the invention. As shown in FIGS. 15 and 17, the activation mechanism 470 may be contained in an end portion 419 of the housing 414 that may be secured to the larger housing, such as by appropriate fasteners, such as screws 472. Turning to FIGS. 17 and 19A-19E, the activation mechanism implements the button slide 480 that is configured to mount and slide in the housing 414. Button slide 480 is activated by a bias element 482 in the housing, in the form of a coil spring, that biases the button slide upwardly in the housing as illustrated in FIGS. 19C and 19E to secure the slide. This provides tactile feedback as described. The button slide 480 also incorporates a button portion 484 that is engaged by the hand of the user as shown in FIG. 20A, 20B to activate the device. The button element is biased upwardly by the spring or other bias element to secure the slide. The button element is pressed against the bias force of the spring or other bias element to release the slide when the device is activated.

For engagement with slide 418, the button slide 480 has a slot 490 formed therein. The slide 418 has a latch end 492 that passes through the slot 490 and includes a notch 494 that is selectively engaged by a catch portion of the button slide. Referring to FIGS. 18B and 19C, when the device 410 is the primed or ready position, the button slide is biased to its upmost position. The slide catch portion 496 is held in the notch 494 of the slide 418. This locks the slide and prevents the slide, and the plunger 420, from extending to the activated position under the bias of spring 430. This is considered a primed position of the device 410.

To activate the device, such as when the needle is in tissue (FIG. 20A), the activation area 417 is engaged by the finger or thumb of the user to engage the button portion 484 of the button slide and press the button slide downwardly against the bias of spring 482. The button slide moves down and slides the catch portion 496 out of the notch 494 of the slide 418 as shown in FIGS. 18C and 19D. This frees the slide 418 to now move under the bias of spring 430 to the activated position. The latch end 492 includes a surface 540 proximate to the notch 494 that is sloped or chamfered for the catch portion 496 to follow when the button slide is depressed. As seen if FIG. 19C as the button slide catch portion 496 moves downwardly, it will eventually reach the surface 540. Under the bias of the spring 430 pulling the slide rearwardly, the catch portion 496 will then ride up the surface 540 to a flat surface 542 on the slide. This further pulls the button slide and button portion 484 further down toward area 417. In one embodiment, the amount of travel of the slide and plunger in the initial activation phase of pushing the button will be controlled so that the catch portion 496 will sit on flat surface 542 while the syringe body and plunger create the vacuum as shown in FIG. 19D. The device is partially activated. In accordance with a feature of the invention, the button slide 480 and latch end 492 are configured and dimensioned so that as the catch portion sits on surface 542, the top of the button portion will sit below a top surface 548 of the activation area 417 as shown in area 550 of FIG. 19D. Therefore, when the device is initially activated, the user will have the tactile feel of the button portion going away from their finger or disappearing into the housing. The device is in the vacuum stage and waiting for the needle to enter tissue space, such as vascular space. All of that time in partial activation, the button latch spring 482 is compressed and acting or biasing upwardly on the button latch.

As noted herein, upon initial or partial activation, the slide 418 and plunger 420 will move some distance (e.g. 1 cm) under spring bias or another bias force to set up a drawing pressure or vacuum in the syringe cavity 415 through the stopper 421 of the plunger. If the insertion end of the needle 432 is blocked, such as by tissue, the slide 418 will stop with the button slide resting on the slide and will be continuously biased by spring 430 until the needle penetrates into a vascular space to allow blood or fluid to move in the needle and allow the slide and plunger to move to the full activated position, as shown in FIGS. 18D and 20B, to move wire 424. As shown in FIG. 19A, the slide catch portion 496 and latch end 492 might include sloped or chamfered surfaces 521, 523 that are complementary and allow the slide catch portion 496 to ride up or down the latch end 492. When the slide and plunger are in the fully activated position, the catch portion 496 slides down off of surface 542 and slides down surface 521 to then be completely released from the latch end of the slide as shown in FIG. 19D. At this point, the button element is again biased upwardly when the slide is released to provide tactile feedback to a user. That is, the release of the slide moves it away from the button slide as shown in FIG. 19E and allows the spring 482 to expand and drive the button slide 480 to snap back upwardly toward area 417. This then drives or snaps the button portion 484 of button slide 480 upwardly and back above the top surface 548 of area 417 to provide a tactile feedback to the user that the needle has entered tissue space or whatever target space is the goal for placement of the wire. In that way, the user may know that full activation is achieved without having to look at the device or see blood flow in the syringe body. The button portion 484 might be pointed or textured to increase the tactile feel of the button moving in and out of the housing.

The tactile feedback for the various stages of the process provided by the invention is a significant advantage if the user is looking at a monitor, such as an ultrasound monitor or other screen, and cannot watch the device. To that end, the latch end 492 of the slide 418 may be configured to modify or amplify the feedback provided by the moving button latch. For example, referring to FIG. 19F, the latch end 492 may use a longer more gradually sloping surface 560 (as opposed to surface 540) for the slide catch portion 496 to ride on when the device is initially activated by pushing the button portion 484 and moved out of notch 494. The catch portion comes out of the notch and rides up the gradual slope. The user will feel the tactile withdrawing of the button latch as it slides up sloping surface 560 The latch end 492 of FIG. 19E does not have a surface 521. Rather, the end surface 562 is abrupt and so the snap back of button slide 480 and feedback to the user is more abrupt or severe. As may be appreciated, the latch end shape and the surfaces of the slide may be adjusted to adjust the type and severity of feedback as the button slide moves thereon. With a flat end surface, to prime the device, the button latch may need to be pushed in as the slide is pushed in to compress the spring.

To that end, the activation mechanism 470 is configured to provide passage for both the wire 424 as well as fluid into the syringe cavity 415. Referring to FIG. 18A, an internal bore of needle 432 progresses into the bore 435 of needle base 433 and into a bore 453 of housing outlet 450. This allows the passage of both the wire 424 and fluid into the housing 414. The wire 424 passes through an opening 481 formed in the button slide 480 and then through a bore 504 and out of the housing to area 451 for manual engagement by the user. The wire then passes back into the housing 414 at a bore 502 to engage with the wire advancing mechanism 434 and ultimately the pocket 436 in slide 418. Referring to FIGS. 17 and 19A, the housing forms a bore 500 that communicates with cavity 415 for the syringe to draw blood therein in operation of device 410. The syringe body sits below the plane of the needle and wire as shown in FIG. 18A. Therefore, bore 500 is below bore 453 that is in line and in the plane with the needle and wire. For diverting and directing blood and fluid between bore 453 and bore 500, as seen in FIG. 18B, a gasket 510 is positioned to span between the bores and direct the fluid flow from bore 453 down and into bore 500 and the syringe body. As seen in FIG. 17, the gasket 510 includes an opening 511 that aligns with the bore 500 to allow fluid to pass through the gasket. As shown in FIG. 18B, the wire 424 passes through gasket 510 and the gasket seals around the wire to prevent blood or fluid from leaving the housing 412. In one embodiment, the gasket is made of a flexible material that may be punctured by wire 424 to allow it to pass through to needle 432. When the device is being assembled and the wire put into position through the needle and into the housing it passes through bore 453 and may penetrate the gasket 510 on the way to bore 504 and out of the housing. The gasket seals around the wire. A spacer plate 512 is positioned with gasket 510 and against button slide 480 to keep gasket 510 in alignment with the bore 500. As shown in FIGS. 17 and 18B, the spacer plate 512 also includes an opening 513 for passage of the wire therethrough and through an opening in the button slide to exit the housing to be exposed in area 451 as shown in FIG. 18B.

In use and operation, device 410 can start in the fully extended or activated position with the wire positioned therein as shown in FIGS. 18A, 19A. The spring is fully extended. A wire 424 may be loaded into the housing, such as through the needle, gasket and housing, and through wire advancing mechanism 434 to engage slide 418. Referring to FIG. 18B, the slide includes a channel 431 along its length to capture and guide the wire 424 to pocket 436. The wire can sit on the channel and the end will fall into the pocket 436 when the device is primed. To move the device to the primed position, the slide and plunger may be pushed in to compress spring 430 as shown by arrow 520 in FIG. 18A. The slide 418 moves in channel 419 with latch end 492 moving toward the latch mechanism 470 of the housing. The latch end 492 will eventually engage the button slide 480 and particularly slide catch portion 496. As shown in FIG. 19A, the slide catch portion 496 and latch end 492 include the sloped or chamfered surfaces 521, 523 that are complementary and allow the slide catch portion 496 to ride down latch end 492 so that the catch portion is below the latch end as shown in FIG. 19B. This also draws the button portion 484 of button slide 480 downwardly in activation area 417. As the slide 418 moves further, the catch portion 496 will eventually arrive at the notch 494 of the latch end 492 and slide into the notch 494 as shown in FIG. 19C and FIG. 18B. At this stage, the spring is compressed and the slide and plunger device are locked in position by the button slide 480. The device is primed for use and biased by compressed spring 430 to move the slide and plunger in the direction of arrow 522. With the latch end 492 slid into the notch 494, the button portion 484, under bias of spring 482 returns to an upward or exposed position to be ready for engagement as shown.

To activate the device 410, such as when the device needle 432 is placed in tissue as shown in FIG. 20A, the button portion 484 may be pressed down by a user as shown in FIGS. 18B and 19C to again take the catch portion 496 out of the notch 494. The biased slide is again free to move to the activated position as shown in FIGS. 18C and 19E. Depending on if the needle has cleared the tissue and entered vascular space as shown in FIG. 20B, the device 410 may remain in the partially activated state as shown in FIGS. 18C and 19D. The vacuum is drawn and the tissue blocking the needle keeps the device partially activated as the needle is manipulated and positioned in the tissue. The button slide, and particularly the catch portion 496 rests on the slide surface 542 which keeps it depressed with respect to activation area 417 as shown in FIG. 19D. Once, the needle is able to draw blood and fluid, the spring 430 moves the slide and plunger further rearwardly from the needle to the fully activated position as shown in FIG. 18D to drive wire 424 into the vascular space in accordance with the invention as shown in FIGS. 20B and 19E.

FIG. 21 illustrates an alternative embodiment of the invention wherein the slide 418a is formed together with or as an extension of the syringe plunger, such that the slide moves as one piece with the syringe plunger. The spring 430 acts on the slide/plunger element 418a similarly to acting on the separate elements as discussed herein and the device 410 operates the same as described.

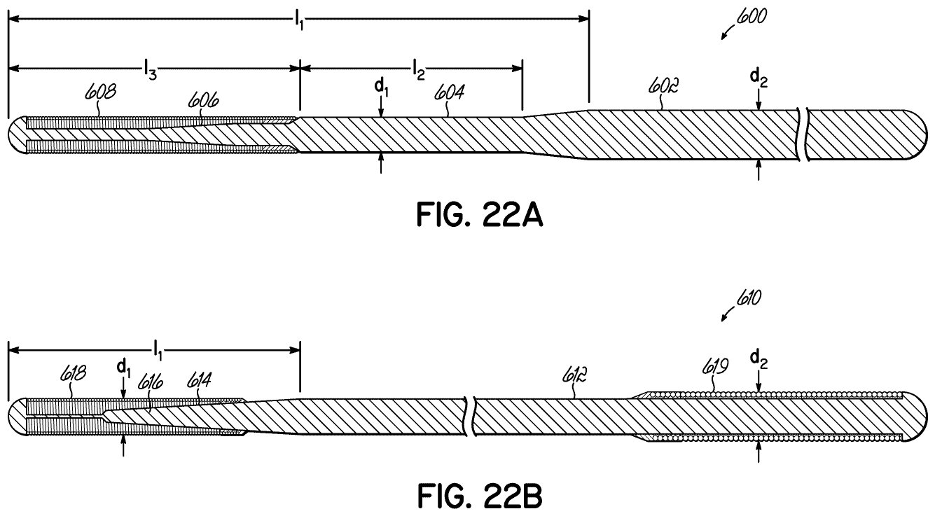

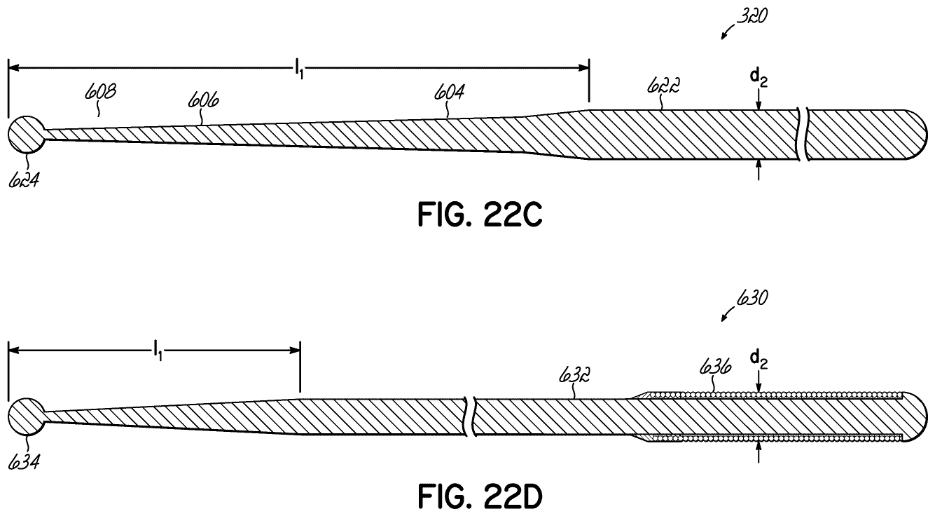

In accordance with another feature of the invention, the wire 424 may have a tapered tip section that cooperates with the needle to allow free blood flow around the wire within the needle in use. The back end may then be thicker to allow manipulation and guidance and to interface with and guide a catheter. To that end FIG. 22 shows a basic wire having a tip section 580 that tapers to a smaller diameter d₁ from a rear section 582 that has a larger diameter d₂. The smaller diameter will be smaller than the lumen or bore through the needle to enhance blood or fluid flow through the needle. FIGS. 22A-22D illustrate other alternative wires that may be used in the invention. For example, FIG. 22A illustrates a 0.018 inch wire 600 that may be used with a 20 gauge or 21 gauge needle. The wire 600 may have a nitinol core 602 having a diameter d₂ of 0.018 inches that tapers down to a diameter d₁ of around 0.012 inches at the tip that is inside the needle. Wire 600 may have an overall length of 45 cm with a taper length l₁ of around 14.5 cm. The tapered length includes a solid section 604 of length 12 of around 7.5 cm and a coiled tip section 606 of length l₃ of around 4.0 cm that is further tapered down for flexibility but includes a stainless steel or other metal coil 608 to maintain the diameter d₁ dimension.

FIG. 22B illustrates 0.032 inch wire 610 that may be used with an 18 gauge needle. The wire 610 may have a nitinol core 612 with an overall diameter d₂ of 0.032 inches that also includes a coil 619 at the backend taper that brings the diameter up to 0.032 inches. The wire may taper down to a solid core diameter d₁ of around 0.020 inches along core 612 and presented at the tip 614 that is inside the needle. Wire 610 may have an overall length of 60-70 cm with a taper length l₁ of around 4-7 cm. The tapered length includes a solid section 616 that is further tapered down for flexibility but includes a stainless steel or other metal coil 618 to maintain the diameter d₁ dimension. A full 0.032 core might be too stiff so the backend coil 619 is used to get to that 0.032 dimension with added flexibility.

The solid or bare core of the wires (as opposed to the spring tip or rear end of the wire) is the section that passes through the gasket 510 in the assembly and arrangement of the wire in the device. This maintains the suction, since coils may allow around them.

FIGS. 22C and 22D illustrate wires that do not use coils at the tips. Rather they taper throughout the length to a small outer diameter and terminate in ball tip or a coil. FIG. 22C illustrates a 0.018 inch wire 620 that may be used with a 20 gauge or 21 gauge needle. The wire 620 may have a nitinol core 622 having a diameter d₂ of 0.018 inches that gradually tapers down to a significantly smaller diameter. The tip may gradually taper down over the length l₁ of around 14.5 cm to a tip 624. The tip 624 may be a wire curled on itself or have a ball shaped tip as illustrated. Wire 620 may have an overall length of 45 cm with a taper length 11 of around 14.5 cm.

FIG. 22D illustrates 0.032 inch wire 630 that may be used with an 18 gauge needle. The wire 610 may have a nitinol core 632 with an overall diameter d₂ of 0.032 inches that also includes a coil 636 at the backend taper that brings the diameter up to 0.032 inche. The wire may taper down to a significantly smaller diameter. Wire 630 may have an overall length of 60-70 cm with a taper length l₁ of around 4-7 cm. The tip may gradually taper down over the length l₁ of around 14.5 cm to a tip 634. The tip 634 may be a wire curled on itself or have a ball shaped tip as illustrated. The wire 630 might also include a similar coil 636 at the backend. A full 0.032 core might be too stiff so the backend coil 636 is used to get to that 0.032 dimension with added flexibility.

The device described herein may be made compatible with MRI machines or other imaging modalities. Also the mechanism may be used with various needle guiding technologies. The mechanism of the invention for wire advancement may also be used also with a vascular access robot.

While a particular needle is shown for device 410, other needle structures, as disclosed herein, like a splitable needle may also be used with device 410 in accordance with the invention.

While the present invention may be used for placing a wire as a guide wire for placing central venous catheters, large arterial access, or for peripheral or smaller arterial and venous access, it may be used for other purposes and techniques as well. For example, it may be used to place pigtail chest tubes, pigtailed drains for fluid collection, for thoracentesis and paracentesis and for other uses where a Seldinger technique is employed or where a wire or catheter is placed in a body. It may also be used for, but not limited to, placement of wires for angiography, central venous line placement, arterial line placement, image guided percutaneous gastrostomy tube placement, small bore chest tube placement, insertion of pacemaker leads, insertion of central venous pressure monitors, cardiac ablation procedures, percutaneous drain placement, PICC placement, insertion of implantable cardioverter-defibrillators, REBOA (resuscitative balloon occlusion of the aorta), endovascular dialysis fistula creation, and other endovascular procedures (EVAR, TEVAR, TAVR, thromboembolectomy, endovascular stent placement, thrombolysis), for example. The wire placed may be used as a guide wire to guide other elements into position or the wire may be the element left in the workspace for the medical procedure.

While the present invention has been illustrated by a description of various embodiments and while these embodiments have been described in some detail, it is not the intention of the inventors to restrict or in any way limit the scope of the appended claims to such detail. Thus, additional advantages and modifications will readily appear to those of ordinary skill in the art. The various features of the invention may be used alone or in any combination depending on the needs and preferences of the user.

What is claimed is:

1. A device for accessing a tissue workspace and delivering a wire into the tissue workspace, the device comprising:
a housing providing a syringe body;
a plunger movable in the syringe body;
a slide configured for coupling with the syringe plunger for moving together with the syringe plunger between a forward position and a retracted position when the device is activated;
a needle configured for coupling with the housing and having at least one bore for guiding a proximal end of the wire into the workspace;
the slide configured for coupling with a distal end of the wire for moving the distal end and advancing the proximal end of the wire forwardly in the needle when the slide moves together with the syringe plunger from the forward position to the retracted position;
a bias force element coupled between the housing and the slide, the bias force element configured for biasing the slide toward the retracted position when the device is activated.

2. The device of claim 1 further comprising a button element movable in the housing to interact with the slide, the button element securing the slide in the forward position and configured for being engaged to release the slide when the device is activated to move to the retracted position.

3. The device of claim 1 wherein the bias force element includes a coil spring, the coil spring configured for being compressed when the slide and syringe plunger are moved to the forward position, the coil spring configured for pushing the slide toward the retracted position when the device is activated.

4. The device of claim 1 wherein the bias force element includes a coil spring, the coil spring configured for being extended when the slide and syringe plunger are moved to the forward position, the coil spring configured for pulling the slide toward the retracted position when the device is activated.

5. The device of claim 1 further comprising a wire advancing mechanism configured for translating the movement of the wire distal end for advancing the proximal end of the wire forwardly in the needle when the wire holding structure and the syringe plunger move together from the forward position to the retracted position.

6. The device of claim 5 wherein the wire advancing mechanism includes a loop guide for reversing the direction of the wire movement and translating the movement of the wire distal end for advancing the wire proximal end.

7. The device of claim 5 wherein the wire advancing mechanism has a distal end positioned to receive the wire from the slide and a proximal end for guiding the proximal end of the wire toward the needle.

8. The device of claim 2 further comprising a bias element acting on the button element, the button element being biased upwardly to secure the slide and being configured to be pressed against a bias force of the bias element to release the slide when the device is activated.

9. The device of claim 8 wherein the button element is biased upwardly when the slide is released to provide tactile feedback to a user that the device is activated.

10. The device of claim 2 wherein the button element includes a catch portion that fits in a notch of the slide to secure the slide in the forward position, the catch portion moving out of the notch when the button element is engaged to release the slide.

11. The device of claim 1 wherein the slide and plunger are a unitary element for moving together between a forward position and a retracted position when the device is activated.

12. The device of claim 1 further comprising a roller positioned on the housing rearwardly of the needle and configured for selectively rolling toward and away from the needle, the roller configured for engaging the wire for moving the wire in the needle when the user moves the roller.

13. The device of claim 1 further comprising a wire, the wire having a tapered end that tapers to a smaller diameter, the tapered end configured for fitting into the needle.

14. A device for accessing a tissue workspace and delivering catheter into a tissue workspace, the device comprising:
a housing providing a syringe body;
a plunger movable in the syringe body;
a slide configured for coupling with the syringe plunger for moving together with the syringe plunger between a forward position and a retracted position when the device is activated;
a wire;
a needle configured for coupling with the housing and having at least one bore for guiding a proximal end of the wire into the workspace;
a catheter positioned on the needle;
the slide configured for coupling with a distal end of the wire for moving the distal end and advancing the proximal end of the wire forwardly in the needle when the slide moves together with the syringe plunger from the forward position to the retracted position;
a bias force element coupled between the housing and the slide, the bias force element configured for biasing the slide toward the retracted position when the device is activated;
the catheter configured for being released from the needle and slid along the wire into the tissue workspace.

15. The device of claim 14 wherein the wire moves through a portion of the housing for advancing the proximal end of the wire forwardly in the needle, the wire including a stop structure thereon configured for being captured with the housing for coupling the wire with the housing.

16. The device of claim 14 further comprising a button element movable in the housing to interact with the slide, the button element securing the slide in the forward position and configured for being engaged to release the slide when the device is activated to move to the retracted position.

17. The device of claim 14 wherein the bias force element includes a coil spring, the coil spring configured for being one of compressed or extended when the slide and syringe plunger are moved to the forward position, the coil spring configured for moving the slide toward the retracted position when the device is activated.

18. The device of claim 16 further comprising a bias element acting on the button element, the button element being biased upwardly to secure the slide and being configured to be pressed against a bias force of the bias element to release the slide when the device is activated.

19. The device of claim 18 wherein the button element is biased upwardly when the slide is released to provide tactile feedback to a user that the device is activated.

20. The device of claim 14 further comprising a roller positioned on the housing rearwardly of the needle and configured for selectively rolling toward and away from the needle, the roller configured for engaging the wire for moving the wire in the needle when the user moves the roller.
